SEQUENCE LISTING (37 sequences)

SEQ ID NO: 1 - DNA, 26 bp, Artificial Sequence
  Synthetic oligonucleotide primer sequence
ataggatggttcatgccgcccatgca

SEQ ID NO: 2 - DNA, 27 bp, Artificial Sequence
  Synthetic oligonucleotide primer sequence
tggggatgaactacatgtgtaacagtt

SEQ ID NO: 3 - DNA, 24 bp, Artificial Sequence
  Auxiliary oligonucleotide
tgttacacatgtagttcatcccca

SEQ ID NO: 4 - DNA, 26 bp, Artificial Sequence
  Auxiliary oligonucleotide
ataggatggttcatgccgcccatgca

SEQ ID NO: 5 - DNA, 17 bp, Artificial Sequence
  Synthetic test sequence
atgcaggaactgttaca

SEQ ID NO: 6 - DNA, 27 bp, Artificial Sequence
  Synthetic oligonucleotide primer sequence
gatggatgacggaggttgtgaggcgct

SEQ ID NO: 7 - DNA, 26 bp, Artificial Sequence
  Synthetic oligonucleotide primer sequence
ataggatggcagcgctcatggtgggg

SEQ ID NO: 8 - DNA, 24 bp, Artificial Sequence
  Auxiliary oligonucleotide
gcctcacaacctccgtcatccatc

SEQ ID NO: 9 - DNA, 26 bp, Artificial Sequence
  Auxiliary oligonucleotide
ataggatggcagcgctcatggtgggg

SEQ ID NO: 10 - DNA, 27 bp, Artificial Sequence
  Synthetic oligonucleotide primer sequence
ataggatgacggaacagctttgaggtg

SEQ ID NO: 11 - DNA, 26 bp, Artificial Sequence
  Synthetic oligonucleotide primer sequence
ataggatgccaggacaggcacaaaca

SEQ ID NO: 12 - DNA, 24 bp, Artificial Sequence
  Auxiliary oligonucleotide
ctcaaagctgttccgtcatcctat

SEQ ID NO: 13 - DNA, 26 bp, Artificial Sequence
  Auxiliary oligonucleotide
ataggatgccaggacaggcacaaaca

SEQ ID NO: 14 - DNA, 30 bp, Artificial Sequence
  Synthetic target sequence
cagctttgaggtgcgtgtttgtgcctgtcc

SEQ ID NO: 15 - DNA, 70 bp, Artificial Sequence
  Padlock probe
gcacctcaaagctgcgcatcccatcagatagcgagtcgacgtgaggatgtacgtggacaggcacaaacac

SEQ ID NO: 16 - DNA, 30 bp, Artificial Sequence
  Synthetic target sequence
cagctttgaggtgcctgtttgtgcctgtcc

SEQ ID NO: 17 - DNA, 24 bp, Artificial Sequence
  Auxiliary oligonucleotide
atgggatgcgcagctttgaggtgc

SEQ ID NO: 18 - DNA, 24 bp, Artificial Sequence
  Auxiliary oligonucleotide
tgtgcctgtccacgtacatcctca

SEQ ID NO: 19 - DNA, 15 bp, Artificial Sequence
  Product nucleic acid
gaggtgcgtgtttgt

SEQ ID NO: 20 - DNA, 35 bp, Artificial Sequence
  Synthetic oligonucleotide primer sequence
nnnnnnnnnnnnnnnngagtcnnnnnnnnnnnnnn

SEQ ID NO: 21 - DNA, 34 bp, Artificial Sequence
  Synthetic oligonucleotide primer sequence
nnnnnnnnnnnnnnnnggatgnnnnnnnnnnnnn

SEQ ID NO: 22 - DNA, 63 bp, Artificial Sequence
  Target strand sequence
attagatgaccctgccttgtcgaaactccacgcacaaacacggacaggaccctctctggccgc

SEQ ID NO: 23 - DNA, 20 bp, Artificial Sequence
  Synthetic oligonucleotide primer sequence
atgtacagctttgaggtgcg

SEQ ID NO: 24 - DNA, 19 bp, Artificial Sequence
  Synthetic oligonucleotide primer sequence
tgtttgtgcctgtcagtga

SEQ ID NO: 25 - DNA, 63 bp, Artificial Sequence
  Complementary-target strand sequence
taatctactgggacggaacagctttgaggtgcgtgtttgtgcctgtcctgggagagaccggcg

SEQ ID NO: 26 - DNA, 14 bp, Artificial Sequence
  Synthetic oligonucleotide primer sequence
gcacctcaaagctg

SEQ ID NO: 27 - DNA, 15 bp, Artificial Sequence
  Synthetic oligonucleotide primer sequence
gacaggcacaaacac

SEQ ID NO: 28 - DNA, 70 bp, Artificial Sequence
  Padlock probe sequence
gcacctcaaagctgtacatcctgccagattgcgagttgaatcacggatggtcactgacaggcacaaacac

SEQ ID NO: 29 - DNA, 42 bp, Artificial Sequence
  Target sequence to be amplified by LCR
gacggaacagctttgaggtgcgtgtttgtgcctgtcctggga

SEQ ID NO: 30 - DNA, 19 bp, Artificial Sequence
  Synthetic oligonucleotide primer sequence
tctgactgtaccaccatcc

SEQ ID NO: 31 - DNA, 22 bp, Artificial Sequence
  Synthetic oligonucleotide primer sequence
tttcttgcggagattctcttcc

SEQ ID NO: 32 - DNA, 23 bp, Artificial Sequence
  Synthetic oligonucleotide primer sequence
ccgacggaacagctttgaggtgc

SEQ ID NO: 33 - DNA, 23 bp, Artificial Sequence
  Synthetic oligonucleotide primer sequence
agtcccaggacaggcacaaacac

SEQ ID NO: 34 - DNA, 21 bp, Artificial Sequence
  Synthetic oligonucleotide primer sequence
gcacctcaaagctgttccgtc

SEQ ID NO: 35 - DNA, 21 bp, Artificial Sequence
  Synthetic oligonucleotide primer sequence
gtgtttgtgcctgtcctggga

SEQ ID NO: 36 - DNA, 73 bp, Artificial Sequence
  Padlock probe sequence
gcacctcaaagctgttccgtccctgccagattgcgagttgaatcacggatggtcccaggacaggcacaaacac

SEQ ID NO: 37 - DNA, 15 bp, Artificial Sequence
  Synthetic oligonucleotide detection probe
cctatagtgagtcgt

(19) United States
(12) Patent Application Publication
(10) Pub. No.: US 2005/0118616 A1
(43) Pub. Date: Jun. 2, 2005

(54) Amplification of target nucleotide sequence without polymerase chain reaction

(76) Inventors: Tadashi Ryan Kawashima, San Diego, CA (US); Erik Holmlin, San Diego, CA (US); Donald M. Crothers, Northford, CT (US); Honghua Zhang, San Diego, CA (US); Chunnian Shi, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/914,114

(22) Filed: Aug. 9, 2004

Related U.S. Application Data
(63) Continuation-in-part of application No. PCT/US03/25544, filed on Aug. 14, 2003.
(60) Provisional application No. 60/404,195, filed on Aug. 16, 2002.

(51) Int. Cl. C12Q 1/68; C12P 19/34

(52) U.S. Cl. 435/6; 435/91.2

(57) ABSTRACT

The present disclosure relates to methods for amplifying nucleic acids and methods for detecting the amplified nucleic acid products. Nucleic acid amplification and detection can occur in solution or on a solid support.

### RELATED APPLICATIONS

This application a continuation-in-part of and claims priority under 35 U.S.C. § 120 to PCT Application No. PCT/US03/25544, entitled AMPLIFICATION OF TARGET NUCLEOTIDE SEQUENCE WITHOUT POLYMERASE CHAIN REACTION, filed Aug. 14, 2003, which claims priority under 35 U.S.C. § 119(e) to U.S. Provisional Application No. 60/404,195, entitled AMPLIFICATION OF TARGET NUCLEOTIDE SEQUENCE WITHOUT POLYMERASE CHAIN REACTION, filed Aug. 16, 2002. The disclosure of each of the above-listed priority applications is incorporated herein by reference in its entirety.

### FIELD OF THE INVENTION

The present disclosure relates generally to the field of biotechnology. More specifically, the present disclosure relates to methods for detecting nucleic acid sequences that have been amplified without the use of polymerase chain reaction.

### BACKGROUND OF THE INVENTION

Amplification of Target Sequences

A number of methods have been developed for amplification of target nucleotide sequences in nucleic acid templates. These include the polymerase chain reaction (PCR), rolling circle amplification (RCA), ligase chain reaction (LCR), self-sustained sequence replication (3SR), nucleic acid sequence based amplification (NASBA), and strand displacement amplification (SDA).

Current methods of PCR amplification involve the use of two primers which hybridize to the regions flanking the target nucleotide sequence, such that DNA replication initiated at the primers will replicate the target nucleotide sequence. By separating the replicated strands from the template strand with a denaturation step, another round of replication using the same primers can lead to many-fold amplification of the target nucleotide sequence.

Rolling circle amplification (RCA) is an isothermal amplification method in which a circularizable single-stranded probe is hybridized to a template such as RNA or denatured DNA at regions flanking the target nucleotide sequence, the strand is circularized using primer extension and/or ligation, sequences in the circle are then selectively amplified, and optionally, non-circular products are removed by digestion.

Ligase chain reaction (LCR) is an amplification method in which, typically, two ligatable pairs of oligonucleotides known as LCR primers or probes, are employed in excess over the target nucleotide sequence, where one pair of the LCR primers are hybridizable to the other. Template containing target nucleotide sequence is first denatured if double-stranded, the LCR primers are allowed to hybridize to their respective complementary strands, and the hybridized primers are then ligated by DNA ligase to form LCR products. The LCR products are then dissociated from the template and can function as target nucleotide sequences themselves. By repeated cycles of hybridization and ligation, amplification of the target nucleotide sequence is achieved. The process of LCR is described in the literature, including U.S. Pat. No. 5,573,907, EP 0 439,182 B1, and EP 0 320,308 B1, among others.

Linear and Nonlinear Amplification of Target Sequences

Amplification of target sequences may be carried out in linear or non-linear mode, for example as described in EP 0971039 to Rabanni et al. Linear amplification of target sequences may be used when a starting mixture contains a large number of copies of a target sequence. Generally, linear amplification utilizes a single initial primer, probe, or other nucleic acid construct to carry out the amplification process.

Non-linear amplification of target sites is often used when the number of copies of a target sequence present in the starting mixture is small. Non-linear amplification results in exponential growth in the number of gene copies present. PCR and RCA, especially RCA in the branching mode, can be used effectively in the non-linear amplification mode. (Lizardi et al., 1998, Nature Genetics 19:225-232)

Generation of Single Stranded DNA

Many amplification methods generate double-stranded amplification products, while many applications require single-stranded DNA molecules containing the target sequence. Double-stranded DNA can be converted to single-stranded DNA by separating the strands or by removing one strand of the duplex. Strands of a duplex can be separated by thermal or chemical methods of disrupting interstrand bonds. Removing one strand allows recovery of the desired strand and elimination of its complement. One strategy for selectively removing one strand of a DNA duplex is to use exonuclease digestion, preferably 5′→3′ exonuclease digestion, where one strand is protected from attack by the exonuclease.

For example, U.S. Pat. No. 5,518,900 to Nikiforov et al. describes modifying one of two PCR primers used for amplification by incorporating phosphorothioate nucleotide derivatives in the 5′ end of the modified primer, rendering it resistant to exonuclease digestion. After amplifying target sequences using PCR, the double-stranded amplification product is subjected to exonuclease digestion. The unprotected strand is preferentially digested by a 5′→3′ exonuclease, leaving a single-stranded product consisting of the other strand.

In an alternate approach, Shchepinov et al. uses branched PCR primers that are resistant to 5′-exonuclease digestion, with the result that exonuclease digestion of the double-stranded amplification products gave single strands protected from digestion by the exonuclease-resistant branched primers. (Shchepinov et al., 1997, Nuc Acids Res 25:4447-4454) Disadvantages of this method are that branched primers are difficult to synthesize and the resulting PCR products are branched.

Another approach to generating single-stranded DNA uses phosphorylation of the 5′ end of one strand of a double-stranded amplification product to produce a preferred lambda exonuclease substrate. (Higuchi et al., 1989, Nuc Acids Res 25: 5685) This method allows selective degradation of the phosphorylated strand and recovery of the nonphosphorylated strand.

Generation of Short Single-Stranded DNA Molecules:

Short single-stranded DNA molecules of defined sequence and length are needed for applications such as arrays, where the desirable size range is about 45 nucleotides or less. Although methods for generating single-stranded DNA molecules are known in the art, these methods do not necessarily generate small molecules of 45 nucleotides or less. For example, the methods discussed above for generating single-stranded DNA do not provide short single-stranded DNA molecules of defined sequence and length. U.S. Pat. No. 5,518,900 to Nikiforov et al. teaches methods for generating single-stranded DNA molecules from double-stranded PCR amplification products, but the resulting PCR products are typically longer than 45 nucleotides. The method of Shchepinov et al. produces branched PCR products that are typically longer than 45 nucleotides. (Shchepinov et al., 1997, Nuc Acids Res 25:4447-4454) Likewise, the method of Higuchi et al. yields single-stranded DNA products that are not in the desired size range. (Higuchi et al. 1989, Nuc Acids Res 17: 5865)

Shaw and Mok disclose cleaving single-stranded DNA into fragments by interaction with a specially designed oligodeoxyribonucleotide adaptor and the class-IIN restriction endonuclease, XcmI. (Shaw and Mok, 1993, Gene 133:85-89) After hybridizing to the target DNA and addition of XcmI, template DNA is specifically cleaved to near completion; however, hairpin structures on the template close to the hybridization site reduce the efficacy of cleavage.

### SUMMARY OF THE INVENTION

The invention described herein is directed to methods for generating a single-stranded DNA molecule of defined sequence and length, where the method includes amplification, conversion, and trimming steps. In accordance with one aspect of the invention, amplification of a template having at least one target nucleotide sequence is directed by one or more primers having at least one exogenous nucleotide sequence not present in the target nucleotide sequence, where the amplification step generates amplification products with at least one target nucleotide sequence and at least one exogenous nucleotide sequence introduced by the primer. In accordance with another aspect of the invention, a conversion step may be performed. When the amplification step generates double-stranded amplification products, the method includes a conversion step wherein each double-stranded amplification product is converted to a single-stranded amplification product. When the amplification step generates single-stranded amplification products, the conversion step is not required. In accordance with another aspect of the invention, the single-stranded amplification product is trimmed to generate a single-stranded DNA molecule of defined sequence and length.

In accordance with one aspect of the invention, polymerase chain reaction (PCR) is used for the amplification step to produce double-stranded amplification products. In one embodiment, multiplex PCR may be used. The amplification step can be carried out in linear or non-linear mode. The template for amplification may be genomic DNA, cDNA, or RNA.

In accordance with another aspect of the invention, rolling circle amplification (RCA) is used for the amplification step. In various embodiments, RCA may produce double-stranded or single-stranded amplification products. In one embodiment, RCA in the linear mode is used to generate single-stranded amplification products. The amplification step can be carried out in linear or non-linear mode. The template for amplification may be genomic DNA, cDNA, or RNA, including mRNA.

In one embodiment, primers for the amplification step may have an addressable ligand such as biotin attached to the primer. In another embodiment, exogenous nucleotide sequence introduced by primers used in the amplification step may contain self-complementary sequences that form hairpin structures. These self-complementary sequences that form hairpin structures may contain at least one restriction enzyme recognition site for a restriction enzyme involved in the trimming step, and suitable restriction enzymes include Type II restriction enzymes such as EcoRI, or Type IIS restriction enzymes such as FokI.

In another embodiment, exogenous nucleotide sequence(s) introduced by primers include sequence(s) that can form a recognition site for a restriction enzyme involved in said trimming step, where the restriction enzyme recognition site is formed upon addition of at least one auxiliary oligonucleotide. Suitable restriction enzymes include Type II restriction enzymes such as EcoRI, or Type IIS restriction enzymes such as FokI. In another embodiment, the auxiliary oligonucleotide includes at least one sequence having an addressable ligand such as biotin attached.

In accordance with another aspect of the invention, the conversion step may be carried out by digesting one strand of a double-stranded amplification product using a 5′→3′ exonuclease such T7 or lambda exonuclease, where the amplification product includes at least one target nucleotide sequence and at least one exogenous nucleotide sequence introduced by a primer during the amplification step. In a preferred embodiment, the exogenous nucleotide sequence introduced by a primer includes modified nucleotides that confer resistance to digestion using 5′→3′ exonuclease, for example where the nucleotides are phosphorothioate derivatives. In another preferred embodiment, the exogenous nucleotide sequence introduced by a primer includes modified nucleotides that confer sensitivity to digestion using 5′→3′ exonuclease, for example where the modified nucleotides are phosphorylated.

In accordance with another aspect of the invention, a method is provided for generating a single-stranded DNA molecule of defined sequence and length which avoids the exonuclease step and a requirement for auxiliary oligonucleotides. The method includes amplifying a template containing at least one target nucleotide sequence, where the amplification is directed by at least one primer having at least one exogenous nucleotide sequence not present in the target nucleotide sequence, generating a plurality of double-stranded amplification products having at least one target nucleotide sequence and at least one exogenous nucleotide sequence introduced by at least one primer, then nicking each double stranded amplification product at one end of a defined sequence and cleaving the double stranded amplification product at the other end of a defined sequence to generate a DNA molecule of defined sequence and length, and finally, separating the single stranded DNA molecule of defined sequence and length from the remainder of the amplification product that includes its complement and the primer duplexes of the amplification product. The single stranded DNA molecule of defined sequence and length can be recovered for further use. In accordance with one aspect, the single stranded DNA molecule of defined sequence and length is separated from the remainder of the amplification product by heating under conditions that allow the single stranded DNA molecule of defined sequence and length to separate from its complement while leaving the primer duplexes of the amplification product intact. In accordance with another aspect, the primers include an addressable ligand attached to the primer. In one embodiment, the addressable ligand is biotin, and the remainder of the amplification product can be removed by attachment to magnetic beads carrying streptavidin that binds to biotin labels attached to the 5′ end of at least one primer.

In accordance with the methods of the present invention, the single-stranded DNA molecule of defined sequence and length generated by the present invention may be between 10 and 100 nucleotides, or between 10 and 50 nucleotides in length. In one embodiment, the single-stranded DNA molecule of defined sequence and length is 15 nucleotides in length. In another embodiment, the single-stranded DNA molecule of defined sequence and length is about 17 nucleotides in length. In yet another embodiment, the single-stranded DNA molecule of defined sequence and length is about 21 nucleotides in length. In yet another embodiment, the single-stranded DNA molecule of defined sequence and length is about 30 nucleotides in length. It will be appreciated, however, that single-stranded nucleic acids, including single-stranded DNA molecules, can range in length from about 4 nucleotides, about 5 nucleotides, about 6 nucleotides, about 7 nucleotides, about 8 nucleotides, about 9 nucleotides, about 10 nucleotides, about 11 nucleotides, about 12 nucleotides, about 13 nucleotides, about 14 nucleotides, about 15 nucleotides, about 16 nucleotides, about 17 nucleotides, about 18 nucleotides, about 19 nucleotides, about 20 nucleotides, about 21 nucleotides, about 22 nucleotides, about 23 nucleotides, about 24 nucleotides, about 25 nucleotides, about 26 nucleotides, about 27 nucleotides, about 28 nucleotides, about 29 nucleotides, about 30 nucleotides, about 31 nucleotides, about 32 nucleotides, about 33 nucleotides, about 34 nucleotides, about 35 nucleotides, about 40 nucleotides, about 45 nucleotides, about 50 nucleotides, about 55 nucleotides, about 60 nucleotides, about 65 nucleotides, about 70 nucleotides, about 75 nucleotides, about 80 nucleotides, about 85 nucleotides, about 90 nucleotides, about 95 nucleotides, about 100 nucleotides, or more than 100 nucleotides in length.

Another aspect of the present invention is directed to methods for identifying an organism or individual using some or all of the following steps: 1) obtaining template having at least one target nucleotide sequence; 2) amplifying the template in an amplification reaction directed by at least one primer having an exogenous nucleotide sequence not present in the target nucleotide sequence; 3) generating amplification products having at least one target nucleotide sequence and at least one exogenous nucleotide sequence introduced by a primer; 4) converting double-stranded amplification products to single-stranded amplification products; trimming each single-stranded amplification product to generate a single-stranded DNA molecule of defined sequence and length; 5) determining the mass or nucleotide sequence of each single-stranded DNA molecule of defined sequence and length; and 6) using at least one mass or nucleotide sequence determination of at least one single-stranded DNA molecule of defined sequence and length to identify at least one organism or individual. In accordance with another aspect of the invention, it is understood that in some embodiments, the amplification step can produce single-stranded amplification products. In one embodiment, mass spectroscopy may be used to determine the mass or nucleotide sequence of each single-stranded DNA molecule of defined sequence and length. In another embodiment, a multiplicity of individuals or organisms is identified by this method.

In accordance with yet another aspect of the invention, ligation is used for the amplification step. Preferably, ligase chain reaction (LCR) is used for the amplification step. In accordance with still another aspect of the invention, LCR is used as a pre-amplification step prior to one or more amplification steps. In one embodiment, LCR is repeated using temperature cycling to generate an exponential amplification of the target nucleotide sequence.

Another aspect of the invention is directed to methods for generating a single-stranded DNA molecule of defined sequence and length by obtaining single-stranded template comprising at least one target nucleotide sequence and contacting the template with a plurality of oligonucleotide LCR primers, where at least one pair of the LCR primers is designed to hybridize to the target nucleotide sequence on the template such that the 5′ end of one of said pair of LCR primers hybridizes adjacent to the 3′ end of the other of said pair of LCR primers, and where at least one LCR primer of the pair of LCR primers includes exogenous nucleotide sequence not present in the target nucleotide sequence. The template and LCR primers are then incubated with ligase under conditions that promote adjacent hybridization of at least one pair of LCR primers to the target nucleotide sequence on the template and ligation of any adjacent hybridized pair of LCR primers to form at least one LCR product that includes sequence complementary to the target nucleotide sequence and exogenous nucleotide sequence. The LCR product is then dissociated from the template, the hybridization and ligation steps are repeated as desired, and the LCR products are recovered.

In one embodiment, single-stranded template is obtained by denaturing double-stranded DNA to generate single-stranded template from the target strand that contains at least one target nucleotide sequence, and single-stranded template from the complementary-target strand. The double-stranded DNA may be genomic DNA.

In one preferred embodiment, the LCR primers include LCR primers having sequences complementary to variant sequences of the target nucleotide sequence, such that only LCR primers complementary to the variant sequence present in the target strand template will hybridize to the template and form at least one LCR product having sequence that is complementary to the variant sequence present in the template. In another preferred embodiment, the LCR primers include at least a pair of LCR primers designed to hybridize adjacently to the target strand template, where one LCR primer of the pair includes exogenous nucleotide sequence 3′ to sequence complementary to a portion of the target nucleotide sequence and one LCR primer of said pair includes exogenous nucleotide sequence 5′ to sequence complementary to the remaining portion of the target nucleotide sequence, such that the LCR product includes exogenous nucleotide sequence flanking the sequence complementary to the target nucleotide sequence.

In another even more preferred embodiment, each LCR primer complementary to the target nucleotide sequence on the target strand includes exogenous nucleotide sequence complementary to a portion of the backbone of a padlock probe, where the LCR product then includes 5′ and 3′ exogenous nucleotide sequence complementary to a portion of the backbone of said padlock probe, where the sequence complementary to a portion of the backbone flanks sequence complementary to the target nucleotide sequence. In yet another preferred embodiment, the LCR product is incubated with at least one padlock probe in linear form that includes backbone sequence complementary to the 5′ and 3′ exogenous nucleotide sequence of the LCR product, under conditions that promote hybridization of the padlock probe in linear form to the LCR product such that the 5′ end of the padlock probe is adjacent to the 3′ end of the padlock probe and the 5′ and 3′ ends are ligated to form a circularized padlock probe. In yet another embodiment, DNA polymerase can be added to the padlock probe-LCR product complex under conditions that permit rolling circle amplification (RCA) of the target nucleotide sequence using said circularized padlock probe, where RCA generates a single-stranded DNA molecule including multiple copies of the target nucleotide sequence. In another preferred embodiment, at least one exogenous nucleotide sequence introduced by at least one LCR primer includes sequences involved in post-amplification cleavage of the single-stranded DNA molecule. RCA using a padlock probe and target LCR product.

In one embodiment, the LCR primer can include at least one sequence having an addressable ligand attached, where the addressable ligand is preferably biotin. In another embodiment, the at least one exogenous nucleotide sequence introduced by at least one LCR primer can include a site for enzymatic digestion of said single-stranded DNA molecule, including sequences involved in trimming DNA molecules produced by enzymatic digestion of the single-stranded DNA molecule. Sequences involved in trimming DNA molecules produced by enzymatic digestion of the single-stranded DNA molecule may include self-complementary sequences that form hairpin structures, and may preferably include self-complementary sequences that form hairpin structures having at least one restriction enzyme recognition site for a restriction enzyme involved in the trimming step. Preferably, restriction enzymes involved in the trimming step may be a Type II or Type IIS restriction enzyme, including EcoRI or FokI. Sequences involved in trimming may include sequences that form at least one restriction enzyme recognition site for a restriction enzyme involved in the trimming step upon addition of at least one auxiliary oligonucleotide. At least one auxiliary oligonucleotide may include at least one sequence having an addressable ligand attached, preferably biotin.

In another preferred embodiment, the LCR primers include at least one pair of LCR primers that adjacently hybridize to the target nucleotide sequence and at least one pair of LCR primers that adjacently hybridize to the complementary-target nucleotide sequence, under conditions that promote hybridization and ligation of each pair of LCR primers, such that, after dissociating each said LCR product from the template, complementary portions of each said LCR product hybridize to form a double-stranded LCR product. Preferably, one strand of the double-stranded LCR product can be digested using a 5′→3′ exonuclease such as T7 or lambda exonuclease. More preferably, the at least one exogenous nucleotide sequence introduced by at least one LCR primer includes modified nucleotides that confer resistance to digestion using 5′→3′ exonuclease or sensitivity to digestion using 5′→3′ exonuclease. Even more preferably, the at least one LCR primer complementary to the complementary-target stranded includes modified nucleotides that confer resistance to digestion using 5′→3′ exonuclease or sensitivity to digestion using 5′→3′ exonuclease. Preferably, the modified nucleotides are phosphorothioate derivatives or phosphorylated.

Another aspect of the present invention provides a method for generating a single-stranded DNA molecule of defined sequence and length by obtaining single-stranded template comprising at least one target nucleotide sequence and contacting the template with a plurality of oligonucleotide LCR primers, where at least one pair of the LCR primers is designed to hybridize to the target nucleotide sequence on the template such that the 5′ end of one of said pair of LCR primers hybridizes adjacent to the 3′ end of the other of said pair of LCR primers, and where at least one LCR primer of the pair of LCR primers includes exogenous nucleotide sequence not present in the target nucleotide sequence. The template and LCR primers are then incubated under conditions that promote adjacent hybridization of at least one pair of LCR primers to the target nucleotide sequence on the template and non-enzymatic ligation of any adjacent hybridized pair of LCR primers to form at least one LCR product that includes sequence complementary to the target nucleotide sequence and exogenous nucleotide sequence. The LCR product is then dissociated from the template, the hybridization and ligation steps are repeated as desired, and the LCR products are recovered.

Additional embodiments of the present invention are described in the numbered paragraphs below:

1. A method for generating a single-stranded nucleic acid product, said method comprising the steps of:
(a) obtaining a plurality of ligase chain reaction (LCR) primers comprising a first pair of LCR primers, wherein at least one LCR primer of said first pair of LCR primers comprises an exogenous nucleotide sequence located adjacent to a nucleotide sequence which is complementary to a portion of a target nucleotide sequence;
(b) hybridizing said first pair of LCR primers with a template nucleic acid that comprises said target nucleotide sequence such that the 5′ end of one primer of said first pair of LCR primers hybridizes to said target nucleotide sequence adjacent to the 3′ end of the other primer of said first pair of LCR primers;
(c) incubating said template nucleic acid and said plurality of LCR primers under conditions that promote ligation of said adjacently hybridized LCR primers thereby forming an LCR product comprising a nucleotide sequence that is complementary to said target nucleotide sequence and adjacent to at least one exogenous nucleotide sequence;
(d) dissociating said LCR product from said template nucleic acid;
(e) hybridizing said LCR product to a padlock probe comprising a backbone nucleotide sequence flanked at the 5′ end by a nucleotide sequence complementary to a portion of said LCR product that is complementary to said target nucleotide sequence and flanked at the 3′ end by a nucleotide sequence complementary to the remainder of said LCR product that is complementary to said target nucleotide sequence, wherein the 5′ end of said padlock probe and the 3′ end of said padlock probe are adjacent each other;
(f) incubating said LCR product hybridized to said padlock probe under conditions to promote ligation of the 5′ end of said padlock probe to the 3′ end of said padlock probe; and
(g) initiating polymerization from the 3′ end of said LCR product thereby generating a single-stranded nucleic acid product comprising a plurality of copies of said target nucleotide sequence.

2. The method of Paragraph 1, wherein said template nucleic acid comprises a single-stranded nucleic acid.

3. The method of Paragraph 1, wherein said template nucleic acid comprises a double-stranded DNA.

4. The method of Paragraph 3, wherein said double-stranded DNA is denatured prior to step (b).

5. The method of Paragraph 4, wherein said double-stranded DNA is a genomic DNA.

6. The method of Paragraph 1, wherein one primer of said first primer pair of LCR primers comprises an exogenous nucleotide sequence located adjacent to the 3′ end of a nucleotide sequence which is complementary to a portion of a target nucleotide sequence and wherein the other LCR primer of said first pair of LCR primers comprises an exogenous nucleotide sequence located adjacent to the 5′ end of a nucleotide sequence which is complementary to a portion of said target nucleotide sequence.

7. The method of Paragraph 1, wherein said first pair of LCR primers comprise LCR primers having sequences complementary to variant sequences of said target nucleotide sequence, such that only LCR primers complementary to said variant sequence present in said target nucleotide sequence hybridize to said template nucleic acid and form at least one LCR product comprising sequence complementary to said variant sequence that is present in said target nucleotide sequence.

8. The method of Paragraph 1, wherein said exogenous nucleotide sequence is complementary to a portion of said backbone of the padlock probe.

9. The method of Paragraph 8, wherein said exogenous nucleotide sequence further comprises sequences involved in post-amplification cleavage of said single-stranded nucleic acid product.

10. The method of Paragraph 9, wherein at least one LCR primer comprises a nucleotide sequence having an addressable ligand attached thereto.

11. The method of Paragraph 10, wherein said addressable ligand is biotin.

12. The method of Paragraph 9, wherein said exogenous nucleotide sequence introduced into said single-stranded nucleic acid product by an LCR primer comprises a site for enzymatic digestion of said single-stranded nucleic acid product.

13. The method of Paragraph 12, wherein said exogenous nucleotide sequence introduced into said single-stranded nucleic acid product by an LCR primer further comprises a sequence involved in trimming nucleic acid molecules produced by said enzymatic digestion of said single-stranded nucleic acid product.

14. The method of Paragraph 13, wherein said sequence involved in trimming said nucleic acid molecules produced by said enzymatic digestion of said single-stranded nucleic acid product comprise a self-complementary sequence that forms a hairpin structure.

15. The method of Paragraph 14, wherein said self-complementary sequence that forms a hairpin structure comprises a restriction enzyme recognition site for a restriction enzyme involved in said trimming step.

16. The method of Paragraph 15, wherein said restriction enzyme involved in said trimming step is a Type II or Type IIS restriction enzyme.

17. The method of Paragraph 16, wherein said Type II restriction enzyme is EcoRI.

18. The method of Paragraph 16, wherein said Type IIS restriction enzyme is FokI.

19. The method of Paragraph 13, wherein said sequence involved in trimming comprises a sequence that forms a restriction enzyme recognition site upon addition of an auxiliary oligonucleotide.

20. The method of Paragraph 19, wherein said restriction enzyme involved in said trimming step is a Type II or Type IIS restriction enzyme.

21. The method of Paragraph 20, wherein said Type II restriction enzyme is EcoRI.

22. The method of Paragraph 20, wherein said Type IIS restriction enzyme is FokI.

23. The method of Paragraph 19, wherein said auxiliary oligonucleotide comprises a sequence having an addressable ligand attached thereto.

24. The method of Paragraph 23, wherein said addressable ligand is biotin.

25. The method of Paragraph 4, further comprising hybridizing a second pair of LCR primers with said template nucleic acid such that said second pair of LCR primers adjacently hybridizes to a nucleotide sequence complementary to the target nucleotide sequence thereby producing nucleic acid complementary to at least a portion of said LCR product.

26. The method of Paragraph 25, wherein said nucleic acid complementary to at least a portion of said LCR product is digested using 5′→3′ exonuclease.

27. The method of Paragraph 26, wherein said 5′→3′ exonuclease is T7 or lambda exonuclease.

28. The method of Paragraph 25, wherein said exogenous nucleotide sequence introduced into said single-stranded nucleic acid product by an LCR primer comprises modified nucleotides that confer resistance to digestion using 5′→3′ exonuclease.

29. The method of Paragraph 28, wherein said modified nucleotides are phosphorothioate derivatives.

30. The method of Paragraph 25, wherein said at least one LCR primer of said second pair of LCR primers comprises an exogenous nucleotide sequence which comprises modified nucleotides that confer sensitivity to digestion using 5′→3′ exonuclease.

31. The method of Paragraph 30, wherein said modified nucleotides are phosphorylated.

32. The method of Paragraph 1, wherein said conditions that promote ligation are enzymatic conditions.

33. The method of Paragraph 1, wherein said conditions that promote ligation are non-enzymatic conditions.

34. A method for generating a single-stranded nucleic acid comprising:
hybridizing a first ligase chain reaction (LCR) primer and a second LCR primer to a target nucleotide sequence of a template nucleic acid such that the 3′ end of said first LCR primer is adjacent to the 5′ end on said second LCR primer, wherein at least one of said first LCR primer and said second LCR primer comprise an exogenous nucleotide sequence which is not complementary to said target nucleotide sequence;
ligating said first LCR primer to said second LCR primer to generate a LCR product;
hybridizing a padlock probe to said LCR product, said padlock probe comprising a nucleotide sequence complementary to at least a portion of said LCR product, wherein said portion includes the 3′ end of said LCR product;
ligating the ends of said padlock probe which is hybridized to said LCR product to one another, thereby generating a circularized padlock probe;
initiating polymerization from the 3′ end of said LCR product which is hybridized to said padlock probe, thereby generating a single-stranded nucleic acid comprising said exogenous nucleic acid sequence.

35. The method of Paragraph 34, wherein said template nucleic acid comprises a single-stranded nucleic acid.

36. The method of Paragraph 34, wherein said template nucleic acid comprises a double-stranded DNA.

37. The method of Paragraph 36, wherein said double-stranded DNA is a genomic DNA.

38. The method of Paragraph 34, wherein said first LCR primer comprises an exogenous nucleotide sequence located adjacent to the 5′ end of a nucleotide sequence which is complementary to a portion of a target nucleotide sequence and wherein the second LCR primer comprises an exogenous nucleotide sequence located adjacent to the 3′ end of a nucleotide sequence which is complementary to a portion of said target nucleotide sequence.

39. The method of Paragraph 34, wherein said LCR primers comprise sequences complementary to variant sequences of said target nucleotide sequence, such that only LCR primers complementary to said variant sequence present in said target nucleotide sequence hybridize to said template nucleic acid and form at least one LCR product comprising sequence complementary to said variant sequence that is present in said target nucleotide sequence.

40. The method of Paragraph 34, wherein said exogenous nucleotide sequence is complementary to a portion of said backbone of the padlock probe.

41. The method of Paragraph 40, wherein said exogenous nucleotide sequence further comprises sequences involved in post-amplification cleavage of said single-stranded nucleic acid product.

42. The method of Paragraph 41, wherein at least one LCR primer comprises a nucleotide sequence having an addressable ligand attached thereto.

43. The method of Paragraph 42, wherein said addressable ligand is biotin.

44. The method of Paragraph 41, wherein said exogenous nucleotide sequence introduced into said single-stranded nucleic acid product by an LCR primer comprises a site for enzymatic digestion of said single-stranded nucleic acid product.

45. The method of Paragraph 44, wherein said exogenous nucleotide sequence introduced into said single-stranded nucleic acid product by an LCR primer further comprises a sequence involved in trimming nucleic acid molecules produced by said enzymatic digestion of said single-stranded nucleic acid product.

46. The method of Paragraph 45, wherein said sequence involved in trimming said nucleic acid molecules produced by said enzymatic digestion of said single-stranded nucleic acid product comprise a self-complementary sequence that forms a hairpin structure.

47. The method of Paragraph 46, wherein said self-complementary sequence that forms a hairpin structure comprises a restriction enzyme recognition site for a restriction enzyme involved in said trimming step.

48. The method of Paragraph 47, wherein said restriction enzyme involved in said trimming step is a Type II or Type IIS restriction enzyme.

49. The method of Paragraph 48, wherein said Type II restriction enzyme is EcoRI.

50. The method of Paragraph 48, wherein said Type IIS restriction enzyme is FokI.

51. The method of Paragraph 45, wherein said sequence involved in trimming comprises a sequence that forms a restriction enzyme recognition site upon addition of an auxiliary oligonucleotide.

52. The method of Paragraph 51, wherein said restriction enzyme involved in said trimming step is a Type II or Type IIS restriction enzyme.

53. The method of Paragraph 52, wherein said Type II restriction enzyme is EcoRI.

54. The method of Paragraph 52, wherein said Type IIS restriction enzyme is FokI.

55. The method of Paragraph 51, wherein said auxiliary oligonucleotide comprises a sequence having an addressable ligand attached thereto.

56. The method of Paragraph 55, wherein said addressable ligand is biotin.

57. The method of Paragraph 36, further comprising hybridizing a third LCR primer and a fourth LCR primer with said template nucleic acid such that said third LCR primer and said fourth LCR primer adjacently hybridize to a nucleotide sequence complementary to the target nucleotide sequence thereby producing nucleic acid complementary to at least a portion of said LCR product.

58. The method of Paragraph 57, wherein said nucleic acid complementary to at least a portion of said LCR product is digested using 5′→3′ exonuclease.

59. The method of Paragraph 58, wherein said 5′→3′ exonuclease is T7 or lambda exonuclease.

60. The method of Paragraph 57, wherein said exogenous nucleotide sequence introduced into said single-stranded nucleic acid product by an LCR primer comprises modified nucleotides that confer resistance to digestion using 5′→3′ exonuclease.

61. The method of Paragraph 60, wherein said modified nucleotides are phosphorothioate derivatives.

62. The method of Paragraph 57, wherein said at least one of said third LCR primer and said fourth LCR primer comprises an exogenous nucleotide sequence which comprises modified nucleotides that confer sensitivity to digestion using 5′→3′ exonuclease.

63. The method of Paragraph 62, wherein said modified nucleotides are phosphorylated.

64. The method of Paragraph 34, wherein said ligating step is an enzymatic ligation.

65. The method of Paragraph 1, wherein said ligating step is a non-enzymatic ligation.

66. A method for generating a single-stranded DNA molecule of defined sequence and length comprising the following steps:
obtaining single-stranded template comprising at least one target nucleotide sequence;
contacting said template with a plurality of oligonucleotide LCR primers, wherein at least one pair of said LCR primers is designed to hybridize to said target nucleotide sequence on said template such that the 5′ end of one of said pair of LCR primers hybridizes adjacent to the 3′ end of the other of said pair of LCR primers, further wherein at least one LCR primer of said pair of LCR primers comprises exogenous nucleotide sequence not present in said target nucleotide sequence;
incubating said template and said plurality of LCR primers with ligase under conditions that promote adjacent hybridization of at least one pair of LCR primers to said target nucleotide sequence on said template and ligation of any said adjacent hybridized pair of LCR primers to form at least one LCR product comprising sequence complementary to said target nucleotide sequence and further comprising exogenous nucleotide sequence;
dissociating said LCR product from said template;
repeating said hybridization and ligation steps as desired; and
recovering said LCR products.

67. A method for detecting a nucleic acid comprising:
hybridizing a first ligase chain reaction (LCR) primer and a second LCR primer to a target nucleotide sequence of a template nucleic acid such that the 3′ end of said first LCR primer is adjacent to the 5′ end of said second LCR primer;
ligating said first LCR primer to said second LCR primer to generate an LCR product;
hybridizing an RCA probe to at least a portion of said LCR product which includes the 3′ end said LCR product;
initiating polymerization from said 3′ end of said LCR product, thereby generating an amplified single-stranded nucleic acid which comprises a plurality of LCR products; and
detecting said amplified single-stranded nucleic acid or fragments thereof.

68. The method of Paragraph 67, wherein at least one LCR primer comprises an exogenous nucleotide sequence which is not complementary to said target nucleotide sequence.

69. The method of Paragraph 68, wherein said exogenous nucleotide sequence is complementary to at least a portion of a nucleotide sequence present in a capture probe.

70. The method of Paragraph 68, wherein said exogenous nucleotide sequence is complementary to at least a portion of a sequence present in a detection probe.

71. The method of Paragraph 67, wherein said RCA probe comprises an exogenous sequence which is not present in said target sequence.

72. The method of Paragraph 71, wherein said exogenous nucleotide sequence is identical to at least a portion of a nucleotide sequence present in a capture probe.

73. The method of Paragraph 71, wherein said exogenous nucleotide sequence is identical to at least a portion of a sequence present in a detection probe.

74. The method of Paragraph 67, wherein said RCA probe is circular.

75. The method of Paragraph 67, wherein said RCA probe is a padlock probe.

76. The method of Paragraph 75, further comprising ligating the ends of said padlock probe to one another, thereby generating a circularized padlock probe.

77. The method of Paragraph 67, wherein detecting the amplified single-stranded nucleic acid comprises coupling one or more detector reagents to said amplified single-stranded nucleic acid and detecting said detector reagent.

78. The method of Paragraph 67, wherein detection the amplified single-stranded nucleic acid comprises detecting an electrical signal indicative of the presence of said amplified single-stranded nucleic acid.

79. The method of Paragraph 78, wherein said detector reagent is a detection probe.

80. The method of Paragraph 79, wherein said detection probe is complementary to a nucleotide sequence that is present in the amplified single-stranded nucleic acid.

81. The method of Paragraph 79, wherein a detector molecule is coupled to said detection probe.

82. The method of Paragraph 81, wherein said detector molecule is fluorescein.

83. The method of Paragraph 78, wherein said detector reagent is a redox enzyme.

84. The method of Paragraph 83, wherein said redox enzyme is HRP.

85. The method of Paragraph 78, wherein said detector reagent is a metal ion.

86. The method of Paragraph 85, wherein said metal ion is ruthenium.

87. The method of Paragraph 67, wherein said template nucleic acid comprises a single-stranded nucleic acid.

88. The method of Paragraph 67, wherein said template nucleic acid comprises a double-stranded DNA.

89. The method of Paragraph 88, wherein said double-stranded DNA is a genomic DNA.

90. The method of Paragraph 88, wherein said first LCR primer comprises an exogenous nucleotide sequence located adjacent to the 5′ end of a nucleotide sequence which is complementary to a portion of a target nucleotide sequence and wherein the second LCR primer comprises an exogenous nucleotide sequence located adjacent to the 3′ end of a nucleotide sequence which is complementary to a portion of said target nucleotide sequence.

91. The method of Paragraph 88, wherein said LCR primers comprise sequences complementary to variant sequences of said target nucleotide sequence, such that only LCR primers complementary to said variant sequence present in said target nucleotide sequence hybridize to said template nucleic acid and form at least one LCR product comprising sequence complementary to said variant sequence that is present in said target nucleotide sequence.

92. A kit comprising:
a chip having a capture probe attached thereto;
a detector reagent; and
instructions for using said detector reagent to detect an amplified target nucleotide sequence attached to said chip via said capture probe.

93. The kit of Paragraph 92, wherein said capture probe is attached to a detection zone.

94. The kit of Paragraph 92, wherein said detector reagent is a detection probe.

95. The kit of Paragraph 94, wherein said detection probe is complementary to a nucleotide sequence that is present in the amplified single-stranded nucleic acid.

96. The kit of Paragraph 94, wherein a detector molecule is coupled to said detection probe.

97. The kit of Paragraph 96, wherein said detector molecule is fluorescein.

98. The kit of Paragraph 92, wherein said detector reagent is a redox enzyme.

99. The kit of Paragraph 98, wherein said redox enzyme is HRP.

100. The kit of Paragraph 92, wherein said detector reagent is a metal ion.

101. The kit of Paragraph 100, wherein said metal ion is ruthenium.

102. The kit of Paragraph 92, further comprising an LCR primer pair.

103. The kit of Paragraph 102, wherein at least one LCR primer of said primer pair comprises an exogenous nucleotide sequence that is complementary to at least a portion of a nucleotide sequence of said capture probe.

104. The kit of Paragraph 102, wherein at least one LCR primer of said primer pair comprises an exogenous nucleotide sequence that is complementary to at least a portion of a nucleotide sequence of a detection probe.

105. The kit of Paragraph 92, further comprising an RCA probe having an exogenous nucleotide sequence that is identical to at least a portion of a nucleotide sequence of said capture probe.

106. The kit of Paragraph 105, wherein said RCA probe is a padlock probe.

107. The kit of Paragraph 105, wherein said RCA probe is circular.

108. The kit of Paragraph 92, further comprising an RCA probe having an exogenous nucleotide sequence that is identical to at least a portion of a nucleotide sequence of a detection probe.

109. The kit of Paragraph 108, wherein said RCA probe is a padlock probe.

110. The kit of Paragraph 108, wherein said RCA probe is circular.

### BRIEF DESCRIPTION OF THE DRAWINGS

FIG. 1. Outline of the method, showing amplification to produce double-stranded molecules, digestion of one strand, and trimming the resulting single strand to the final length.

FIG. 2. A circularizable linear DNA molecule containing at its two ends sequences complementary to a target sequence A is hybridized to the template. If the sequence is designed so that the 3′ and 5′ ends are immediately adjacent, the molecule is circularized by DNA ligase. If a gap remains, it is filled by DNA polymerase, and the molecule is subsequently ligated into a circle. Exogenous sequences in the circularizable molecule are indicated as potential trimming sites. The circularized molecule contains the sequence A′ which is complementary to the target sequence A.

FIG. 3. Linear RCA amplification of the circular molecule. The single-stranded product contains the target sequence A, flanked on both sides by exogenous sequences designed for the trimming reaction.

FIG. 4. Illustration of trimming by restriction digestion at sites formed by addition of auxiliary oligonucleotides complementary to the exogenous sequences in the single-stranded product.

FIG. 5. Illustration of trimming by restriction digestion at hairpin helical sites encoded by the exogenous sequences in the circularizable molecule.

FIG. 6. Illustration of PCR probes used to introduce exogenous sequences that encode restriction sites for the trimming reaction. The double-stranded amplification products is shown.

FIG. 7. Illustration of the use of auxiliary oligonucleotides to provide a double helical substrate for a Type IIS restriction enzyme (e.g., FokI). In this case, the enzyme recognition sequence is encoded in hairpin helical structures that are derived from exogenous sequences in the primers.

FIG. 8. Illustration of trimming by a Type II restriction enzyme (e.g., EcoRI), whose recognition sites are encoded in exogenous sequences in the primers.

FIG. 9. Illustration of ligase chain reaction (LCR) using probes to amplify target nucleotide sequence and introduce exogenous sequences into LCR products.

FIG. 10. Illustration of padlock probe ligation to LCR product containing target nucleotide sequence to form a ligated padlock probe concatenated to the target.

FIG. 11. Illustration of LCR-based RCA, PCR-based RCA, and comparison of the kinetics of each amplification reaction.

FIG. 12A. A denaturing polyacrylamide gel which shows FokI processing of a 55 bp PCR product containing a SNP encoding an S241F mutation in the p53 gene product. The double-stranded 55 bp target nucleotide sequence amplified from gDNA (Lane 1), the single-stranded 55 base target nucleotide sequence after digestion with X exonuclease (Lane 2), the single-stranded 55 base target nucleotide sequence with the 26-mer and 24-mer auxiliary oligonucleotides (Lane 3), and the single-stranded 17 base product of FokI digestion containing the target SNP (Lane 4).

FIG. 12B. An autoradiogram of a non-denaturing polyacrylamide gel showing that the 17 base FokI digestion product of FIG. 12A hybridizes to a synthetic 17 base oligonucleotide complementary to the SNP-containing target sequence.

FIG. 13. An autoradiogram of a native polyacrylamide gel which shows products of an LCR amplification in the presence of synthetic target 42.1 (lane 1), 100 ng human GDNA (lane 2), 800 ng human gDNA (lane 3), 800 ng human gDNA supplemented with sonicated salmon sperm DNA (lane 4), an approximately 540 bp PCR product comprising the target sequence (lane 5), or no target (lane 6).

Additionally, labeled primer 21.7 was run alone (lane 8) or in combination with primer 23.2 (lane 7).

FIG. 14. An autoradiogram of a denaturing polyacrylamide gel which shows the hybridization of a padlock probe to a target RNA and subsequent ligation. Lane 1 contains only single-stranded target RNA whereas lane 2 contains only single-stranded padlock probe. Lane 3 contains an unligated mixture of RNA target and padlock probe. Lane 4 contains RNA target and padlock probe after incubation with T4 DNA ligase.

FIG. 15. A diagram illustrating sequence amplification by branched RCA.

FIG. 16. A diagram illustrating sequence amplification by hyperbranched RCA.

FIG. 17. A diagram illustrating sequence amplification by head-to-tail polymerization.

### DETAILED DESCRIPTION OF THE PREFERRED EMBODIMENT

The present disclosure provides methods for generating single-stranded DNA molecules having defined sequence and length from a template such as genomic DNA, cDNA, or RNA. Advantageously, the methods disclosed and claimed herein enable production of large numbers of single-stranded amplification products containing target nucleotide sequence, which are trimmed to produce single-stranded DNA molecules having defined sequence and length and further, where the entire procedure may, if desired, be performed in a single reaction vessel.

The methods disclosed herein include, but are not limited to, amplification of a template including at least one target nucleotide sequence, using at least one primer or probe having exogenous nucleotide sequence not found in the target nucleotide sequence, generating amplification products including at least one target nucleotide sequence and at least one exogenous nucleotide sequence, converting double-stranded amplification products to single-stranded amplification products if necessary, and trimming single-stranded amplification products to yield single-stranded DNA molecules of defined sequence and length. Advantageously, the methods disclosed herein provide a strategy for generating amplification products including at least one target nucleotide sequence and at least one exogenous nucleotide sequence involved in post-amplification processing of the amplification product. Optionally, at least one exogenous nucleotide sequence, which may include modified bases, is involved in conversion of double-stranded amplification products to single-stranded amplification products. Preferably, at least one exogenous nucleotide sequence is involved in restriction endonuclease-mediated trimming of single-stranded amplification products to generate single-stranded DNA molecules having defined sequence and length.

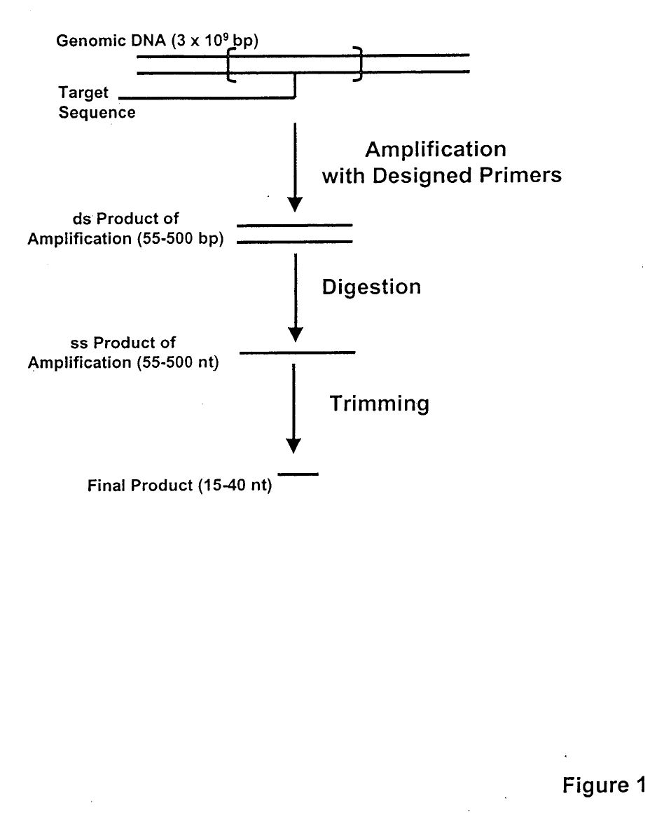

In accordance with one aspect of the present invention, the methods disclosed herein provide amplification methods to generate double-stranded amplification products that are converted to single-stranded amplification products that are then trimmed to yield single-stranded DNA molecules of defined sequence and length (FIG. 1). Advantageously, the methods disclosed herein provide a strategy for generating double-stranded amplification products including at least one target nucleotide sequence and at least one exogenous nucleotide sequence involved in post-amplification processing of double-stranded amplification products, including conversion to single-stranded amplification products and subsequent trimming of single-stranded amplification products. In a preferred embodiment, a double-stranded amplification product comprises two exogenous nucleotide sequences, one at each end of the product, where the exogenous nucleotide sequences are involved in post-amplification processing of double-stranded and/or single-stranded amplification products.

In accordance with another aspect of the present invention, the methods disclosed herein provide amplification methods to generate single-stranded amplification products that are then trimmed to yield single-stranded DNA molecules of defined sequence and length. Advantageously, the methods disclosed herein provide a strategy for generating single-stranded amplification products including at least one target nucleotide sequence and at least one exogenous nucleotide sequence involved in post-amplification trimming of single-stranded amplification products. In a preferred embodiment, a single-stranded amplification product contains one target nucleotide sequence and has two exogenous nucleotide sequences, one at the 3′ and one at the 5′ end of the product, where the exogenous nucleotide sequences are involved in post-amplification processing of single-stranded amplification products. In another preferred embodiment, a single-stranded amplification product contains more than one target nucleotide sequence and each target nucleotide sequence is flanked by exogenous nucleotide sequences, where the exogenous nucleotide sequences are involved in post-amplification processing of single-stranded amplification products.

As used herein, “template” refers to all or part of a polynucleotide containing at least one target nucleotide sequence. As used herein, a “target nucleotide sequence” includes the nucleotide sequence of the final product having defined sequence and length, and may include other nucleotide sequences that are removed during post-amplification processing of the amplification product. Nucleotide sequences that are found in the target nucleotide sequence and later removed may include binding sites (annealing sites) for primers or probes, nucleotides involved in conversion of double-stranded DNA to single-stranded DNA, or sequences useful as recognition and/or cleavage sites for restriction endonucleases. An “exogenous nucleotide sequence” as used herein, refers to a sequence introduced by primers or probes used for amplification, such that amplification products will contain exogenous nucleotide sequence and target nucleotide sequence in an arrangement not found in the original template from which the target nucleotide sequence was copied. As used herein, an “auxiliary oligonucleotide” is a DNA sequence that can be used to create a restriction digestion site by binding to one or more sequences in the single-stranded amplification products. In a preferred embodiment, the auxiliary oligonucleotides are complementary to one or more parts of the single-stranded amplification products, and duplex formation creates a restriction site that enables trimming of the single-stranded amplification product to the final desired size. Auxiliary oligonucleotides and primers may contain chemical modifications to enable the trimmed single-stranded product to be separated from primers and auxiliary oligonucleotides. In a preferred embodiment, the chemical modification is an addressable ligand permitting recovery of a molecule containing the ligand. In a more preferred embodiment, the addressable ligand is a biotin residue.

In accordance with another aspect of the present invention, the template may be any polynucleotide suitable for amplification, where the template contains at least one target nucleotide sequence to be amplified. Suitable templates include DNA and RNA molecules, and may include polynucleotides having modified bases. Preferably, templates are genomic DNA, cDNA, or RNA molecules. In another preferred embodiment, methods disclosed herein can be used to amplify RNA templates directly, without reverse-transcribing the RNA template into cDNA.

In accordance with another aspect of the present invention, the methods disclosed herein provide at least one double-stranded amplification product that is converted to a single-stranded form that is then trimmed to yield at least one single-stranded DNA molecule of defined sequence and length. Advantageously, the method disclosed herein provides a strategy for generating a single-stranded amplification product containing a region having a target nucleotide sequence and at least one exogenous nucleotide sequence that promotes restriction endonuclease-mediated trimming of the single-stranded amplification product to generate a desired single-stranded DNA molecule of defined sequence and length.

Amplification of Polynucleotide Templates

In accordance with one aspect of the invention as disclosed herein, amplification of templates is carried out using well-known methods to generate amplification products including at least one target nucleotide sequence and at least one exogenous sequence involved in post-amplification processing of the amplification product without a significant effect on the amplification itself. Preferably, post-amplification processing includes, but is not limited to, conversion of double-stranded amplification products to single-stranded amplification products, and trimming of single-stranded amplification products to generate a single-stranded DNA molecule of defined sequence and length. Suitable templates include DNA and RNA molecules such as genomic DNA, cDNA, and mRNA. Linear or exponential (nonlinear) modes of amplification may be used with any suitable amplification method, where choice of mode is made by one of skill in the art depending on the circumstances of a particular embodiment. Methods of amplification include, but are not limited to, use of polymerase chain reaction (PCR) and rolling circle amplification (RCA) to amplify polynucleotide templates. Ligase chain reaction (LCR) may also be used as a method of amplification of target nucleotide sequence. LCR may also be used as a method of pre-amplification in combination with other amplification methods including but not limited to PCR and RCA.

Polymerase Chain Reaction

The polymerase chain reaction (PCR) is a method for in vitro amplification of DNA. PCR uses multiple rounds of primer extension reactions in which complementary strands of a defined region of a DNA molecule are simultaneously synthesized by a thermostable DNA polymerase. During repeated rounds of these reactions, the number of newly synthesized DNA strands increases exponentially such that after 20 to 30 reaction cycles, the initial template DNA will have been replicated several thousand-fold or million-fold. Methods for carrying out different types and modes of PCR are thoroughly described in the literature, for example in “PCR Primer: A Laboratory Manual” Dieffenbach and Dveksler, eds. Cold Spring Harbor Laboratory Press, 1995, and by Mullis et al. in patents (e.g., U.S. Pat. Nos. 4,683,195, 4,683,202 and 4,800,159) and scientific publications (e.g. Mullis et al. 1987, Methods in Enzymology, 155:335-350) where the contents of each reference are hereby incorporated by reference in their entireties.

Briefly, PCR proceeds in a series of steps as described below. In the initial step of the procedure, double-stranded template (e.g., genomic DNA or cDNA) is isolated and heat, preferably between about 90° C. to about 95° C., is used to separate the double-stranded DNA into single strands (denaturation step). Cooling to about 55° C. allows primers to adhere to the target region of the template, where the primers are designed to bind to regions that flank the target nucleic acid sequence (annealing step). Thermostable DNA polymerase (e.g., Taq polymerase) and free nucleotides are added to create new DNA fragments complementary to the target region of the template via primer extension (extension step), to complete one cycle of PCR. This process of denaturation, annealing and extension is repeated numerous times, preferably in a thermocycler. At the end of each cycle, each newly synthesized DNA molecule acts as a template for the next cycle, resulting in the accumulation of many hundreds or thousands, or even millions, of double-stranded amplification products from each template molecule.

In multiplex PCR, the assay is modified to include multiple primer pairs specific for distinct target nucleotide sequences of the same template, to allow simultaneous amplification of multiple distinct target nucleotide sequences and generation of multiple distinct single-stranded DNA molecules having the desired nucleotide sequence and length. For example, multiplex PCR can be carried out using the genomic DNA of an organism or an individual as the template, where multiplex PCR will produce multiple distinct single-stranded DNA molecules. The sequence of each distinct single-stranded DNA molecule having the desired nucleotide sequence and length can be determined, for example using mass spectroscopy to rapidly determine sequence, and the results can be used to identify an organism or an individual.

PCR generates double-stranded amplification products suitable for post-amplification processing. If desired, amplification products can be detected by visualization with agarose gel electrophoresis, by an enzyme immunoassay format using probe-based colorimetric detection, by fluorescence emission technology, or by other detection means known to one of skill in the art.

Primers for Amplification

In accordance with one aspect of the present invention, primers are utilized to permit amplification of a template containing a target nucleotide sequence and to introduce additional features into the amplification products. Each primer contains nucleotide sequence that is complementary to a region of target nucleotide sequence in the template, in order for each primer to bind (anneal) to the template. In one embodiment, at least one primer contains exogenous nucleotide sequence 5′ (upstream) of the primer sequence complementary to the primer-binding target nucleotide sequence, with the result that each amplification product contains exogenous nucleotide sequence introduced by the primer. In another embodiment, at least one primer contains exogenous nucleotide sequence 3′ (downstream) of the primer sequence complementary to the primer-binding target nucleotide sequence, with the result that each amplification product contains exogenous nucleotide sequence introduced by the primer. In still other embodiments, two primers are used, where each primer introduces exogenous nucleotide sequence that allow post-amplification manipulation of amplification products without a significant effect on amplification itself. Alternately, more than two primers are used, where each primer introduces exogenous nucleotide sequence that allow post-amplification manipulation of amplification products without a significant effect on amplification itself. For example in addition to a first primer pair comprising a first and second primer, wherein one or both of the first and second primers comprise an exogenous nucleotide sequence, a second primer pair comprising a third and fourth primer, wherein one or both of the third and fourth primers comprise an exogenous nucleotide sequence are used in the methods described herein. Primers for a particular embodiment may be designed by one of skill in the art according to well-known principles, for example as disclosed in Dieffenbach and Dveksler (“General Concepts For PCR Primer Design” in, PCR Primer: A Laboratory Manual, Dieffenbach and Dveksler, eds., supra, the contents of which are hereby incorporated by reference in its entirety.)

In accordance with one aspect of the invention, primer length and sequence are two factors that are relevant in designing the parameters of a successful amplification. The melting temperature (Tₘ) is the temperature at which a nucleic acid duplex “melts” to form two single strands, and Tₘ increases as a function of its length and (G+C) content. Thus, the annealing temperature chosen for a particular embodiment of primer-directed amplification (e.g., PCR or RCA) depends on length and composition of the primer(s). In accordance with one aspect of the present invention, one of skill in the art can practice the methods disclosed herein using any annealing temperature (Tₐ) that permits generating single-stranded DNA molecules having defined sequence and length from genomic DNA or from RNA. Preferably, annealing temperature (Tₐ) is chosen that is about 5° C. below the lowest Tₘ of the pair of primers being used in a particular embodiment.

In some embodiments of the methods disclosed herein, the primers used are sufficiently complex that the likelihood of annealing to sequences other than the chosen target is very low. Preferably, primers used to practice the present invention should be between approximately 17 to 28 bases in length (17-mer to 28-mer). By way of illustration, there is a one-in-four (1/4) chance of finding any base (A, G, C or T) in any given position in a DNA sequence; there is a one-in-sixteen (1/16) chance of finding any dinucleotide sequence (e.g., AG) in a DNA sequence, a one-in-256 (1/256) chance of finding a given four-base nucleotide sequence, and so on. A particular sixteen-base sequence will statistically be present only once in every approximately 4,294,967,296 bases, which is roughly the size of the human or maize genome. An oligonucleotide having at least 17 base pairs will show such specificity for its target sequence that 17-mer or longer primers are routinely used for amplification from genomic DNA or reverse-transcribed RNA (cDNA) of animals and plants. Preferably, base composition should be 50-60% (G+C), and primers should end (3′) in a G or C, or CG or GC to prevent “breathing” of ends and increase efficiency of priming.

Primers suitable for the methods disclosed herein may be “degenerate” primers for use in degenerate PCR to amplify one or more target sequences. Degenerate PCR can be used to find one or more target sequences corresponding to a known protein sequence, or to find homologs, orthologs, or paralogs of a known sequence. The rules of codon usage are relied upon to design a set of degenerate primers that contains primers capable of binding to any of the possible target sequences of interest. Degenerate primers may be generated by synthesizing multiple primers with different nucleotides at positions known to be variable, and/or by introducing the nucleotide inosine at one or more positions known to be variable. Degenerate primers for a particular embodiment may be designed by one of skill in the art according to well-known principles, for example as disclosed in, PCR Primer: A Laboratory Manual, Dieffenbach and Dveksler, eds., supra, the contents of which are hereby incorporated by reference in its entirety.

In accordance with one aspect of the methods disclosed herein, “nested primers” may be included in some embodiments. Nested primers bind to sites on a template that occur within the target sequence of other primer pairs, and to sites on PCR products generated by the other primer pairs. The amplification products produced by nested primers will be smaller than the initial amplification products, and can be identified on the basis of their expected size. Thus, nested primers may be used to increase the specificity of amplification by ensuring that the desired target sequence is amplified to give a product that can be isolated from other amplification products. Nested primers for a particular embodiment may be designed by one of skill in the art according to well-known principles, for example as disclosed in PCR Primer: A Laboratory Manual, Dieffenbach and Dveksler, eds., supra, the contents of which are hereby incorporated by reference in its entirety.

It should be noted that too long a primer length may mean that even high annealing temperatures are not enough to prevent mismatch pairing and non-specific priming. One of skill in the art can determine the range of acceptable primer lengths for a given target region of interest, and can optimize primer design according to the needs of a particular embodiment.

In accordance with another aspect of the present invention, primers used to amplify templates are designed to introduce features into amplification products by means of introducing exogenous nucleotide sequence not found in the target nucleotide sequence. Exogenous sequences may introduce features including, but not limited to, restriction sites, modified nucleotides, promoter sequences, inverted repeats, and other non-template 5′ extensions that allow post amplification manipulation of amplification products without a significant effect on the amplification itself. Preferably, the exogenous sequences are 5′ (“upstream”) of the primer sequence involved in binding to the target nucleotide sequence. In a preferred embodiment, exogenous sequences introduce sites involved in restriction enzyme recognition, binding and cleavage. In an even more preferred embodiment, primers containing inverted repeats or other exogenous sequences are used to introduce self-complementarity at the ends of the amplification product, such that single-stranded amplification products may form secondary structures such as “hairpins” or loops. In another highly preferred embodiment, auxiliary oligonucleotides are added to bind to the exogenous sequence and thereby create the restriction digestion sites needed for trimming to the final size.

Use of Rolling Circle Amplification to Amplify Target Sequences

In accordance with another aspect of the present invention, an isothermal amplification method is used to generate amplification products including a region having the target nucleotide sequence. Preferably, the isothermal replication method is the “rolling circle amplification” (RCA) method. In one preferred embodiment, linear amplification of target sequences is performed using RCA. In another preferred embodiment, non-linear amplification target sequences is performed using RCA. Methods for carrying out RCA are well known in the art, particularly as disclosed by Kool et al. (U.S. Pat. No. 5,714,320), Landegren et al. (U.S. Pat. No. 5,871,921), and Lizardi et al. (Lizardi et al., 1998, Nature Genet 19: 225-232, and U.S. Pat. Nos. 5,854,033, 6,124,120, 6,143,495, 6,183,960, 6,210,884, 6,280,949, 6,287,824) the entire contents of each of which are hereby incorporated by reference in their entireties. Advantageously, RCA is an isothermal method having high specificity and sensitivity for target sequences and a low level of nonspecific background signal, wherein the amount of amplified product is proportional to the number of target sites in the genomic DNA or cDNA template, and optionally wherein a ligation step can be manipulated to carry out allelic discrimination.

The first step in RCA amplification is creation of a circular molecule that contains a sequence complementary to the target nucleotide sequence. A synthetic linear molecule has at its 3′ and 5′ ends sequences of typically 10 to 20 nucleotides that are complementary to the target sequence. In one embodiment there is a gap between the two complementary regions when the linear molecule is hybridized to the target. The gap is filled by primer extension, and the two ends are ligated together to form the circle. In another embodiment, there is no gap, and only the ligation step is employed. In linear RCA amplification, a primer complementary to a sequence on the circularized single strand is added, and a processive polymerase makes a continuous copy of the circle. Alternately, the linear molecule containing target nucleotide sequence and exogenous sequences is circularized when ends are hybridized to a complementary oligonucleotide other than target sequence, and ends are joined by ligation or gap filling as described above. Non-enzymatic methods can also be used to generate circular molecules containing target sequence. The result is a long single-stranded molecule containing many repeats of the target sequence in the circle. The exogenous sequences in the circle are designed such that the long complementary single-stranded product contains restriction sites analogous to those contained in the primers for PCR amplification. Restriction sites are introduced on both sides of the desired single-stranded product. In another preferred embodiment, the restriction sites are created by the addition of an auxiliary oligonucleotides that binds to the exogenous sequence. In non-linear RCA amplification, a second primer complementary to the single strand product of the rolling circle amplification is also added. The products of non-linear RCA amplification are largely double-stranded, and the use of this option requires digestion or removal of one of the strands.

Amplification products generated by RCA may be double-stranded or single-stranded, depending on the amplification strategy chosen for a particular embodiment.

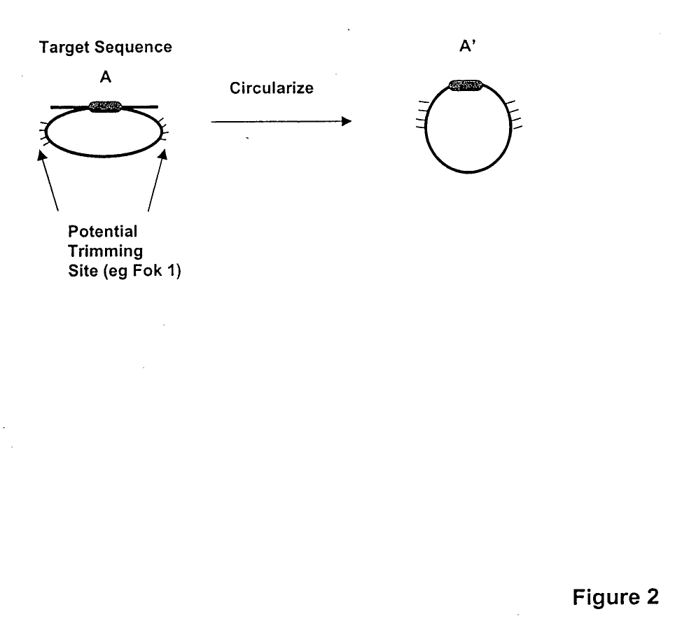

Briefly, a circularizable single strand is hybridized to denatured DNA, then primer extension and/or ligation are used to generate a circular product in the presence of the target sequence, and finally, exonuclease digestion removes non-circular products. In a preferred embodiment, additional sequences are included in the circularizable single strand. In a particularly preferred embodiment, the circularizable molecule is designed and synthesized to include binding sites for restriction endonucleases and/or other enzymes involved in post-amplification manipulations such as trimming amplification products to generate single-stranded DNA molecules of defined sequence and length (FIG. 2).

A ligation step circularizes a specially designed (synthesized) nucleic acid probe molecule, where this step is dependent on hybridization of the probe to a target nucleotide sequence (FIG. 2) and the number of circular probe molecules formed in this step is proportional to the amount of target sequence present in a sample.

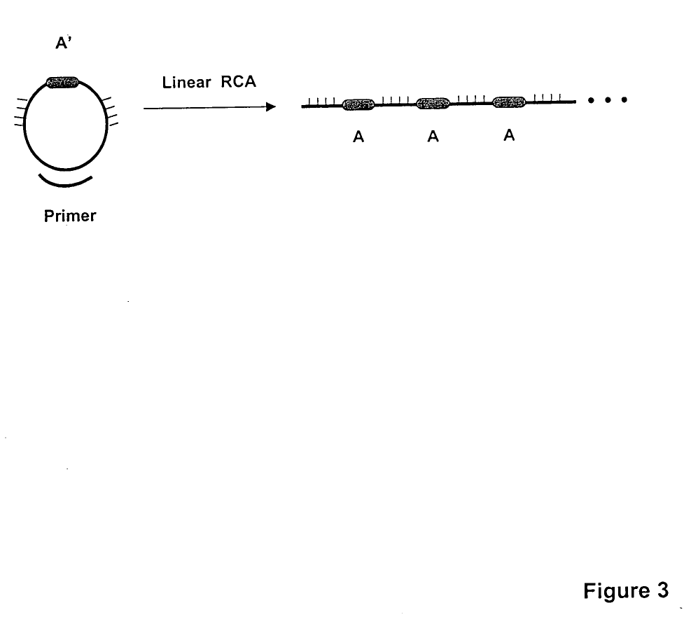

The circular molecule is then amplified using rolling circle replication of the circularized probe, where a single round of amplification using rolling circle replication results in a large amplification of the circularized probe sequences. In one preferred embodiment, the circular molecule is amplified in exponential mode. In another preferred embodiment, the circular molecule is amplified in linear mode (FIG. 3). Advantageously, rolling circle amplification of probes is orders of magnitude greater than a single cycle of PCR or other amplification techniques in which each cycle is limited to a doubling of the number of copies of a target sequence.

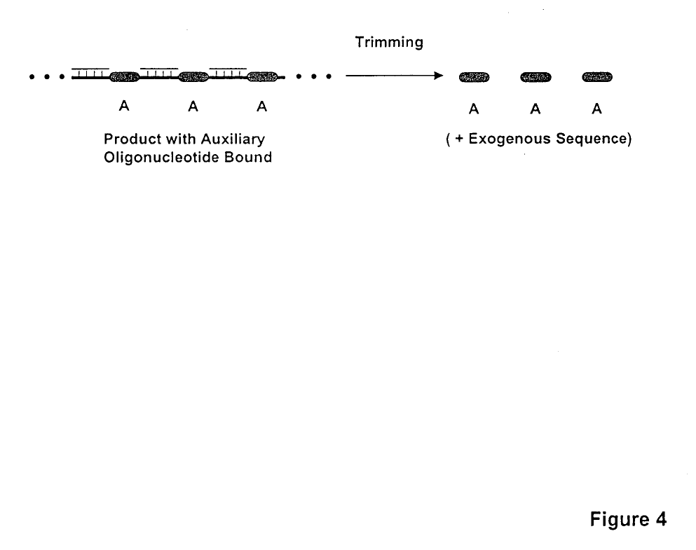

Preferably, the circular molecule is amplified in exponential mode and one of the two primers is protected against 5′-exonuclease digestion using, e.g., 5′-5′ linkage. Alternatively, one primer can be targeted for digestion by 5′ phosphorylation. In such a preferred embodiment, 5′-exonuclease digestion of the product of exponential RCA leaves a protected, long single-stranded molecule capable of binding auxiliary oligonucleotides, and restriction cleavage is carried out as provided in the present disclosure to generate a single-stranded DNA molecule having defined sequence and length (FIG. 4).

Alternately, the circular molecule is amplified in linear mode and the long single-stranded product is trimmed as provided in the present disclosure and auxiliary oligonucleotides are added to provide regions of double-stranded DNA for recognition, binding, and/or cleavage sites for trimming enzymes (FIG. 4).

Optionally, an additional amplification operation can be performed on the DNA produced by RCA. Since the amount of amplified product is directly proportional to the amount of target sequence present in a sample, quantitative measurements of product reliably represent the amount of a target sequence in a sample.

In one embodiment, RCA using two probes (primers) gives rise to linear double-stranded amplification products.

In another embodiment, RCA in a linear mode gives rise to single-stranded amplification products. A circularizable probe can be ligated into a “padlock” configuration using a single primer or gap-filling nucleotides, where the probe is topologically connected to the target through catenation, e.g., as described by Landegren et al. (U.S. Pat. No. 5,871,921), the entire contents of which are hereby incorporated by reference. RCA of a “padlock probe” catalyzed by a strand-displacing DNA polymerase generates a single-stranded amplification product that includes the target nucleotide sequence.

In yet another embodiment, RCA can also be carried out using two primers in a “hyperbranched” mode, known as HRCA, to produce double-stranded amplification products that include the target nucleotide sequence. In multiplex assays, primer oligonucleotides used for DNA replication can be the same oligonucleotides used for all probes.

Probes and Primers for Use in RCA

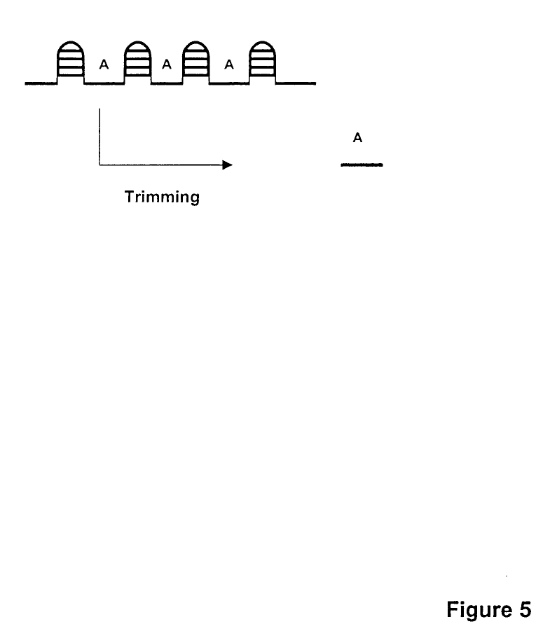

In accordance with another aspect of the present invention, probes and primers used to amplify templates by the RCA method are designed to introduce features into amplification products by means of introducing exogenous nucleotide sequence not found in the target nucleotide sequence. Exogenous sequences may introduce features including, but not limited to, restriction sites, promoter sequences, inverted repeats, and other non-template 5′ extensions that allow post amplification manipulation of amplification products without a significant effect on the amplification itself. Alternately, some modes of RCA produce amplification products having alternating iterations (tandem repeats) of the target nucleotide sequence and the exogenous sequence introduced by probes or primers, such that the exogenous nucleotide sequence is located between copies of target nucleotide sequence. In a preferred embodiment, exogenous nucleotide sequences introduce sites involved in trimming single-stranded amplification products by restriction enzymes in conjunction with auxiliary oligonucleotides. In another preferred embodiment, primers and probes containing inverted repeats or other exogenous sequences are used to introduce self-complementarity at the ends of the amplification product, such that single-stranded amplification products may form secondary structures such as “hairpins” or loops (FIG. 5).

Conversion of Double-Stranded Amplification Products to Single-Stranded DNA

In accordance with another aspect of the present invention, double-stranded amplification products are converted to single-stranded amplification products. Double-stranded amplification products are composed of double-stranded DNA, and single-stranded amplification products are composed of single-stranded DNA, where the DNA strands may include modifications such as phosphorylation, cross-linking groups, or modified bases such as phosphorothioate nucleotide derivatives, as well as other modifications that may be chosen for a particular embodiment by one of skill in the art. Preferably, double-stranded DNA is converted to single-stranded DNA using one or more digestion methods. Advantageously, double-stranded amplification products are digested to provide single-stranded amplification products that can be further manipulated in the same reaction vessel, if desired. In one embodiment, digestion requires that one strand of the double-stranded amplification product contain a chemical modification that either (i) promotes selective digestion or the modified strand, or (ii) inhibits digestion of the modified strand, where such inhibition promotes digestion of the unprotected complementary strand. In another embodiment, digestion of double-stranded amplification products may include simultaneously promoting selective digestion of one strand and inhibiting digestion of the other strand, advantageously to increase the selectively of the digestion step.

In accordance with one aspect of the present invention, at least one primer is resistant to exonuclease digestion, preferably 5′→3′ exonuclease digestion. Digestion-resistant primers or probes can be prepared as described in the art, e.g., in U.S. Pat. No. 5,518,900 issued to Nikiforov et al. Exonuclease-resistant exogenous nucleotide sequences are introduced into amplification products using amplification methods disclosed herein. In one preferred embodiment, PCR or RCA using two primers is carried out in which one primer is resistant to exonuclease digestion. In another preferred embodiment, probes used for RCA can be designed and synthesized to introduce exogenous nucleotide sequence that is resistant to nuclease digestion, preferably 5′→3′ exonuclease digestion.

Suitable enzymes for carrying out digestion of double-stranded amplification products in accordance with the method disclosed herein include T7 exonuclease, lambda (λ) exonuclease, exonuclease m, and other enzymes that may be identified by one of skill in the art as appropriate for a particular embodiment. Enzymes for digesting double-stranded amplification products may be isolated from naturally occurring sources, or may be recombinantly produced.

In one embodiment, T7 exonuclease activity is blocked by introducing a 5′→5′ linkage in one strand, thereby inhibiting digestion of the blocked strand and promoting digestion of the unblocked strand. In another embodiment, T7 exonuclease activity is blocked by incorporating phosphorothioate nucleotide derivatives into one strand, thereby inhibiting digestion of the blocked strand and promoting digestion of the unblocked strand.

In another embodiment, lambda (λ) exonuclease selectively digests one strand of a double-stranded DNA duplex from a 5′ phosphorylated end leaving the complementary strand intact. A 5′ phosphate group is introduced to only one of the two strands during amplification by using one phosphorylated primer and one nonphosphorylated primer, for example as disclosed in Higuchi et al (1989, Nuc Acids Res 17: 5865). The phosphorylated strand is then removed by treatment with lambda exonuclease, generating single-stranded DNA.

In another preferred embodiment, double-stranded amplification products were converted to single-stranded DNA using lambda exonuclease. After amplification of a genomic target sequence using a first primer that is phosphorylated at the 5′ end and a second primer lacking a 5′ phosphate, the 77 base pair double-stranded amplification products were incubated with lambda exonuclease. When the digestion products were separated on an agarose gel, very little 77-nucleotide (nt) single-stranded DNA was seen when no lambda exonuclease was added, and increasing amounts of single-stranded 77-nt DNA was seen with increasing amounts of lambda exonuclease.

In yet another embodiment, incorporation of alphaP-borane 2′-deoxynucleoside 5′-triphosphates (dNT(b)Ps) blocks the action of exonuclease, as described, e.g., by Porter et al. (1997, Nucleic Acids Res. 25:1611-7).

In accordance with another aspect of the invention, non-enzymatic methods may be employed to recover single-stranded DNA from double-stranded amplification products. In one representative embodiment, biotinylated nucleotides are utilized during the amplification step, and biotinylated amplification products can then be captured using a (strept)avidin-coated solid support including but not limited to (strept)avidin-coated beads or surfaces. Once the biotinylated amplification product is bound to the solid support, the sample is subjected to alkaline conditions, or heat, or other conditions suitable to breaking the hydrogen bonds between the two strands. In this embodiment, the nonbiotinylated strand is recovered (eluted) and can be trimmed or otherwise manipulated in accordance with the method disclosed herein.

Trimming Single-Stranded DNA

In accordance with another aspect of the present invention, at least one single-stranded amplification product is trimmed to produce at least one DNA molecule having the desired nucleotide sequence and length, generating a single-stranded DNA molecule of defined sequence and length. Amplification products may be trimmed using restriction endonucleases that cleave at a site distant from their recognition site or may be trimmed using restriction endonucleases that recognize, bind, and cleave at the same site. Preferably, the single-stranded DNA molecule of defined sequence and length generated by trimming is a short molecule having a length from 5 to 50 nucleotides, more preferably a molecule having a length of 10 to 45 nucleotides, even more preferably a molecule having a length of 15 to 40 nucleotides. In accordance with the methods disclosed herein, a single-stranded DNA molecule of defined sequence and length may advantageously be 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides in length.

In accordance with one aspect of the present invention, restriction endonucleases that cleave remotely by recognizing one site and cleaving at another site can be utilized trim the single-stranded amplification product to generate a short DNA molecule of defined sequence and length. Preferably, the remote-acting restriction endonucleases are Class IIS restriction endonucleases that cleave double-stranded DNA at precise distances from the recognition sites. (Szybalski, 1985, Gene 40: 169-173; Podhajska and Szybalski, 1985, Gene 40: 175-182; Sugisaki and Kanazawa, 1981, Gene 16: 73-78) Because of their remote action, these enzymes are also known as “shifters.” (Szybalski, 1985, Gene 40: 169-173) More preferably, the Class IIS restriction endonucleases used to trim DNA include, but are not limited to, BbvI, BbvII, BinI, FokI, HgaI, HphI, MboII, MnlI, SfaNI, TaqII, Tth111II, and MluI. (Szybalski (1985) Gene 40: 169-173; Sugisaki and Kanazawa (1981) Gene 16: 73-78) Advantageously, remote-acting enzymes such as Class IIS restriction endonucleases can be used to trim a DNA molecule even more than when the trimming enzyme binds and cleaves at the same site. Even more advantageously, remote-acting enzymes can be used to generate DNA molecules containing only the desired nucleotide sequence and no unwanted or exogenous sequence.

In one preferred embodiment, FokI is used to trim DNA. FokI was isolated from Flavobacterium okeanokoites (Sugisaki and Kanazawa, 1981, Gene 16: 73-78) FokI uses the a double-stranded recognition site domain containing the sequence GGATG and its complement, and cleaves in a “staggered” pattern 9 and 13 base-pairs away from the recognition site. (Syzbalski, 1985, Gene 40: 169-173; see also, WO0175180) MluI introduces double-strand cleavages at unique sequences that are completely two-fold rotationally symmetric like most type II restriction endonucleases. (Sugisaki and Kanazawa, 1981, Gene 16:73-78)

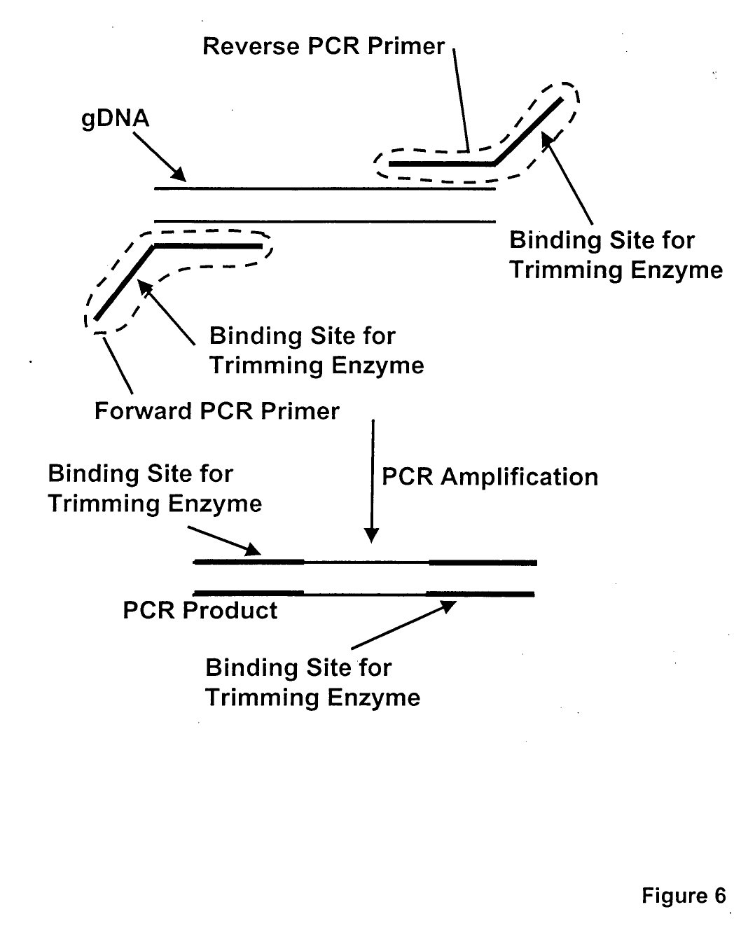

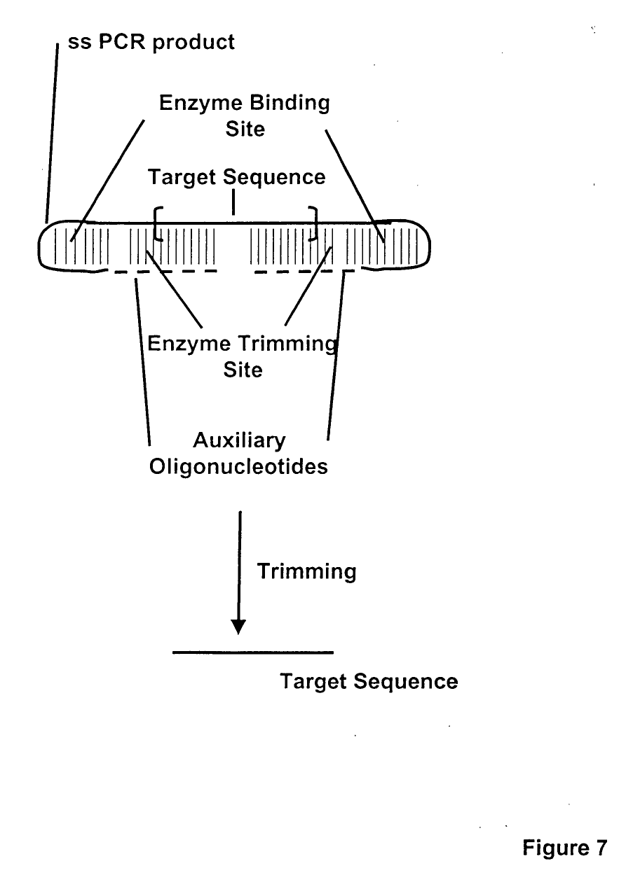

In one preferred embodiment, single-stranded amplification products having terminal hairpin-forming regions are trimmed using FokI. It is necessary to introduce a FokI binding site into the amplification product and provide a double-stranded substrate for FokI binding and cleavage. In an especially preferred embodiment, the binding site is provided as part of the nucleotide sequence of the PCR primers or RCA primers/probes used to amplify templates, to introduce at least one appropriate site into the amplification product, as illustrated in FIG. 6. In one embodiment, primers are designed to produce a double-stranded FokI substrate as follows: forward and reverse primers for PCR have complementary inverted regions such that the single-stranded amplification product generated by digesting a double-stranded amplification product of the amplification would fold back at both ends to form a helix of 8-16 bp containing a FokI binding site, as illustrated by the diagram of FIG. 7. In such an embodiment, auxiliary oligonucleotides that hybridize to the region where cleavage is desired (see FIG. 7) must be supplied in order to provide a region of double-stranded substrate for cleavage. In the present embodiment, FokI cleaves 9 bases from one recognition site and 13 bases from the other. It is understood that such a protocol is not limited to use with FokI, as one of skill in the art could design primers that would introduce exogenous nucleotide sequences including recognition sites for any restriction endonuclease that cleaves at a distance from its recognition site.

In another preferred embodiment, linear primers were used to generate a FokI substrate, preferably when it is not feasible to design primers with tandem repeats as hairpin-forming sequences that generate a complete recognition site. In embodiments using linear primers, primers contain only the top strand sequence of a FokI restriction site, or that of another restriction endonuclease that cleaves at a site distant from its recognition site. In a particularly preferred embodiment, single-stranded amplification products were produced in accordance with the methods of the present invention, and auxiliary oligonucleotides were added that overlap the single strand in two locations, such that one oligonucleotide formed a double strand at the trimming (cleavage) site and another provided the second half of the FokI recognition site. With double-stranded DNA available at recognition and cleavage sites, FokI or a similar restriction endonuclease can trim the DNA molecule to generate a single-stranded molecule of defined length and sequence. It is understood that linear primers for use in amplification, and auxiliary oligonucleotides for use in providing localized double-stranded DNA, could be designed by one of skill in the art in light of the needs, constraints, materials available, or other factors that may be relevant to circumstances of a particular embodiment.

In accordance with another aspect of the invention disclosed herein, restriction endonucleases that bind and cleave at the same site can be used to trim single-stranded amplification products to generate a short single-stranded DNA molecule of defined length and sequence. For example, Type II restriction enzymes bind at a recognition site and cleave within the restriction site; descriptions of the recognition sites and cleavage patterns of Type II enzymes can be found in the art. Preferably, Type II restriction endonucleases are utilized to trim single-stranded amplification products according to the methods disclosed herein. In one preferred embodiment, a restriction enzyme such as EcoR1, is used to trim the amplification product. Primers and/or probes can be designed and synthesized to include a restriction endonuclease binding site, e.g., an EcoR1 binding site.

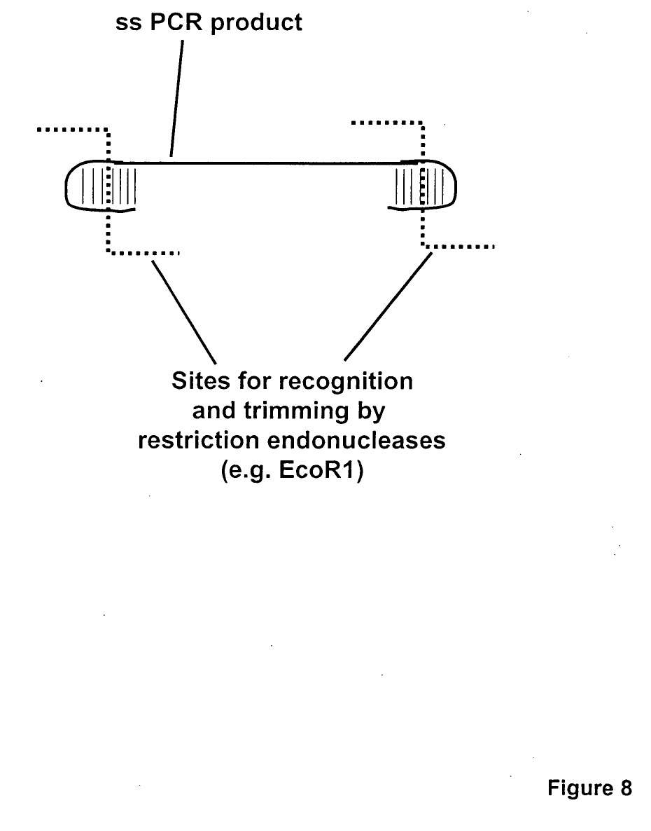

In one preferred embodiment, the primers used in amplification include tandem inverted repeats encoding EcoR1 binding sites, with the result that the ends of the single-stranded amplification product can fold back to form hairpin turns, thereby providing double-stranded DNA at the binding and trimming site. Advantageously, this approach does not require addition of auxiliary oligonucleotides to the single-stranded amplification product (FIG. 8).

In another preferred embodiment, linear primers containing a single copy of the restriction endonuclease recognition site are used in amplification, and auxiliary oligonucleotides including the restriction endonuclease site are added to the single-stranded amplification product to provide a localized region of double-stranded DNA for restriction endonuclease binding and trimming to release a short single-stranded DNA of defined sequence and length.

Use of a Nicking/Cleaving Strategy to Generate Single Stranded DNA Molecules Having Defined Sequence and Length

Another aspect of the invention provides methods for generating single stranded DNA molecules of defined sequence and length wherein the use of exonuclease to release a single strand of DNA and the use of auxiliary oligonucleotides to complete the cleavage site is not necessary. These methods produce an oligomer having the desired nucleotide sequence, generating a single stranded DNA molecule of defined sequence and length from a double stranded amplification product.

In one preferred embodiment, the exonuclease step is avoided by using a nicking enzyme at one end of the defined sequence and cleavage at the other end of the defined sequence, where the defined sequence is contained in a double-stranded amplification product. The oligomer having the defined sequence and length is separated from the remainder of the amplification product, which includes its complement and the primer duplexes of the amplification product, by heating under conditions that allow the oligomer to separate from its complement but leave the primer duplexes intact. Preferably, exogenous sequence introduced by a primer includes an addressable ligand such as biotin attached to the primer, and in one particularly preferred embodiment, the primer complexes are removed by attachment to magnetic beads carrying streptavidin that binds to biotin labels attached to the 5′ end of at least one primer. Example 6 provides an illustrative example of this method.

Amplification of RNA to Generate Single-Stranded DNA Molecules

In accordance with another aspect of the present invention, the methods disclosed and claimed herein may be used to amplify RNA templates to generate short single-stranded DNA molecules of defined sequence and length. RNA may be reversed-transcribed to generate cDNA which may be amplified using any suitable method including, but not limited to, PCR or RCA or LCR. Alternately, RCA may be used to amplify RNA directly.

For procedures that employ PCR, the RNA molecule of interest is reverse-transcribed to provide a cDNA copy suitable for amplification. PCR amplification of a cDNA copy of the RNA of interest generates double-stranded DNA amplification products that must be converted to single-stranded products and trimmed according to aspects of the invention provided in the present disclosure.

In accordance with another aspect of the present invention, RCA may be used to amplify RNA directly, without conversion to cDNA, using RCA in linear or exponential mode. In one embodiment, the primers used to generate the rolling circle include at least one binding site for a trimming enzyme, such that exogenous nucleotide sequence including the binding site is incorporated into the amplification products during the amplification step. Double-stranded amplification products are converted to single-stranded amplification products that are trimmed to generate short single-stranded DNA molecules of defined sequence and length using any of the methods disclosed herein.

As provided in accordance with another aspect of the present invention, RCA can be used in the exponential mode to detect and amplify low copy number messenger RNAs or protein antigens. In a preferred embodiment, DNA microarray applications are developed that exploit signal enhancement by RCA for performing mRNA expression profiling at unprecedented sensitivity. In another preferred embodiment, methods for exponential amplification and in vitro expression of cDNA and genomic DNA fragments are provided, including but not limited to DNA strand displacement reactions that permit isothermal amplification of clones derived from single DNA molecules.

Ligase Chain Reaction

In accordance with another aspect of the present invention, the ligase chain reaction (LCR) can be used to produce a single-stranded product of defined sequence and length. LCR can be used for amplification of target nucleotide sequence without PCR. Alternately, LCR can be used in combination with other methods, including but not limited to PCR or RCA, for amplification of target nucleotide sequence. When used in combination with other amplification methods, LCR can be regarded as a preamplification step, useful for increasing the number of copies of target nucleotide sequence in a mixture.

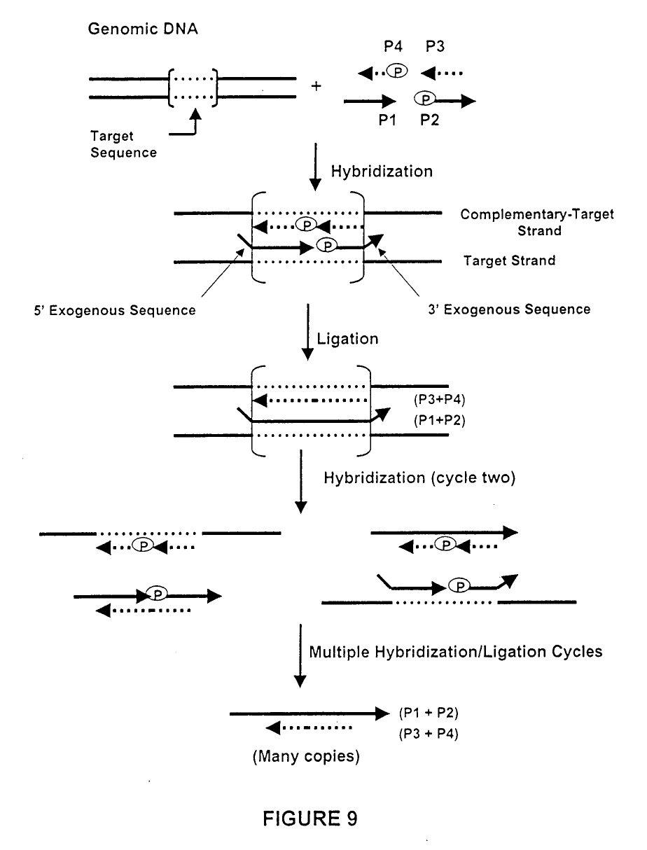

In a typical embodiment, a set of four oligonucleotides known as LCR primers or probes are utilized. In the embodiment illustrated in FIG. 9, the set of four LCR primers have been designated P1, P2, P3, and P4. The design of the LCR primers is such that primer pairs (here, P1/P2 and P3/P4) have sequences that are complementary to both strands of a double-stranded template containing the target nucleotide sequence. The strand containing the target nucleotide sequence is referred to as the target strand; the strand containing the complement of the target nucleotide sequence is referred to as the complementary-target strand. Either the coding or the non-coding strand may be chosen as the target strand; the other strand is the complementary-target strand. Each pair of LCR primers is designed to hybridize adjacently, preferably leaving only a nick that can be ligated. Preferably, each pair of LCR primers is designed to hybridize adjacently at the site of a single nucleotide polymorphism (SNP), leaving only a ligatable nick at the SNP site. Preferably, two of the LCR primers are 5′ phosphorylated and are susceptible to nucleophilic attack by the adjacent primer's 3′ hydroxyl group during ligation. Ligation may be carried out by enzymatic methods using, for example, T4 DNA ligase, (T4 RNA ligase if appropriate), Taq ligase, E. coli DNA ligase, Pfu polymerase or thermal ligase (Tth ligase) as described by Antson et al. (2000, Nuc Acids Res 28:e58). Additionally, any other ligases or enzymes possessing ligase activity that are known in the art may be used. Alternately, ligation may be carried out by nonenzymatic ligation methods, e.g as described by Xu and Kool (1999, Nuc Acids Res 27:875-881), in which case the reaction may be referred to as “nonenzymatic ligation reaction” instead of “ligase chain reaction.”

As used herein, the term “adjacent” as used herein refers to the physical placement of nucleic acid sequence ends relative to one another. For example, in some embodiments of the present invention, the 3′-end of a first nucleic acid and the 5′ end of a second nucleic acid can be bound to a target sequence in such a way that the 3′-end of the first nucleic acid is within at least about 1000 bases of the 5′ end of the second nucleic acid. In such embodiments, the gap between the two nucleic acids can be extended by polymerization and the ends ligated. In some embodiments, the 3′-end of the first nucleic acid is within about 1 base, within about 2 bases, within about 3 bases, within about 4 bases, within about 5 bases, within about 6 bases, within about 7 bases, within about 8 bases; within about 9 bases, within about 10 bases, within about 15 bases, within about 20 bases, within about 25 bases, within about 30 bases, within about 35 bases, within about 40 bases, within about 45 bases, within about 50 bases, within about 60 bases, within about 70 bases, within about 80 bases, within about 90 bases, within about 100 bases, within about 200 bases, within about 300 bases, within about 400 bases, within about 500 bases, within about 600 bases, within about 700 bases, within about 800 bases, within about 900 bases or within about 1000 bases of the 5′ end of the second nucleic acid. In other embodiments, the 3′-end of the first nucleic acid is separated from the 5′ end of the second nucleic acid by only a nick.

As illustrated in FIG. 9, P1 hybridizes adjacent to P2 and P3 hybridizes adjacent to P4, where P2 and P4 are 5′ phosphorylated and susceptible to nucleophilic attack by the 3′ hydroxyl group of P1 and P3 respectively. Preferably, each of the 4 primers P1, P2, P3, and P4 has similar thermal stability when hybridized to its complementary sequence. One simple means of achieving this objective is to adjust the length of each primer as needed to alter its Tₘ so as to bring it to an acceptable value. After LCR is complete, the LCR products P1+P2 and P3+P4 may be recovered and used as desired.

When LCR is used for allelic discrimination, e.g., to detect single nucleotide polymorphisms (SNPs) in a sample, a plurality of primers complementary to variant sequences of the target nucleotide sequence are used. Preferably, the variant primers are distinguished by whether or not the base at the 3′ -end of P1 and P3 is complementary to the SNP base of interest. Because base-mismatch at the ligation site inhibits the ligation reaction, only those primers complementary to the sequence variant(s) present in a sample will be ligated to form a LCR product.

In addition to LCR primers, a reaction mixture contains template having at least one target nucleotide sequence and a thermostable ligase with base recognition properties, as well as any other components deemed necessary, including but not limited to salts, buffers, and blocking agents. Preferably, the template is a sample of genomic DNA from a patient. The reaction mixture is heated to denature double-stranded DNA, the reaction mixture is then cooled to a temperature suitable for hybridization of the LCR primers to the target nucleotide sequence, and template-dependent ligation of the hybridized LCR primers is initiated. The ligation event may be repeated using temperature cycling to generate an exponential amplification of the target nucleotide sequence. Preferably, a genomic DNA sample is heated to about 95 degrees to denature the double-stranded genomic DNA, cooled to a predetermined temperature that will permit optimal hybridization of LCR primers to the genomic template, subjected to template-dependent ligation, and heated again to release LCR product, where the ligation event may be repeated as many times as desired.

If ligation is carried out by non-enzymatic means, ligase may optionally be omitted from the reaction mixture.

In accordance with one aspect of the present invention, LCR primers are designed to permit LCR amplification of a target nucleotide sequence and introduce additional features into the LCR products. In one preferred embodiment, at least one LCR primer contains exogenous nucleotide sequence not found on the template. Exogenous sequence introduced by a LCR primer may allow post-ligation manipulation as described herein including, but not limited to, amplification, trimming, labelling, detection, or capture of LCR products. Example 10 illustrates the use of an exogenous sequence that is present in an LCR primer for attaching a completed LCR product to a solid sufrace. Other embodiments of the present invention also include the use of an exogenous sequence that is present in an LCR primer for detection probe binding. It will be apprecitated, however, that there is no requirement that the LCR primers described herein include an exogenous sequence (see Example 11). LCR products with exogenous sequence may include modified nucleotides such as exonuclease-resistant nucleotides, or nucleotides having an addressable ligand such as biotin.

In an especially preferred embodiment, the set of LCR primers complementary to the target strand are designed to have a different length than the LCR primer set complementary to the complementary-target strand. More preferably, the LCR primer set complementary to the target nucleotide sequence is longer than the LCR primer set complementary to the complementary-target strand, where the primers have additional nucleotides upstream (5′) and/or downstream (3′) to the target nucleotide sequence to which the LCR primers hybridize. Advantageously, the exogenous nucleotide sequence can be at least about 2, 3, 4, 5, 6, 7, 8, 9, 10, 11, 12, 13, 14, 15, 16, 17, 18, 19, 20, 25, 30 or more than 30 nucleotides in length.

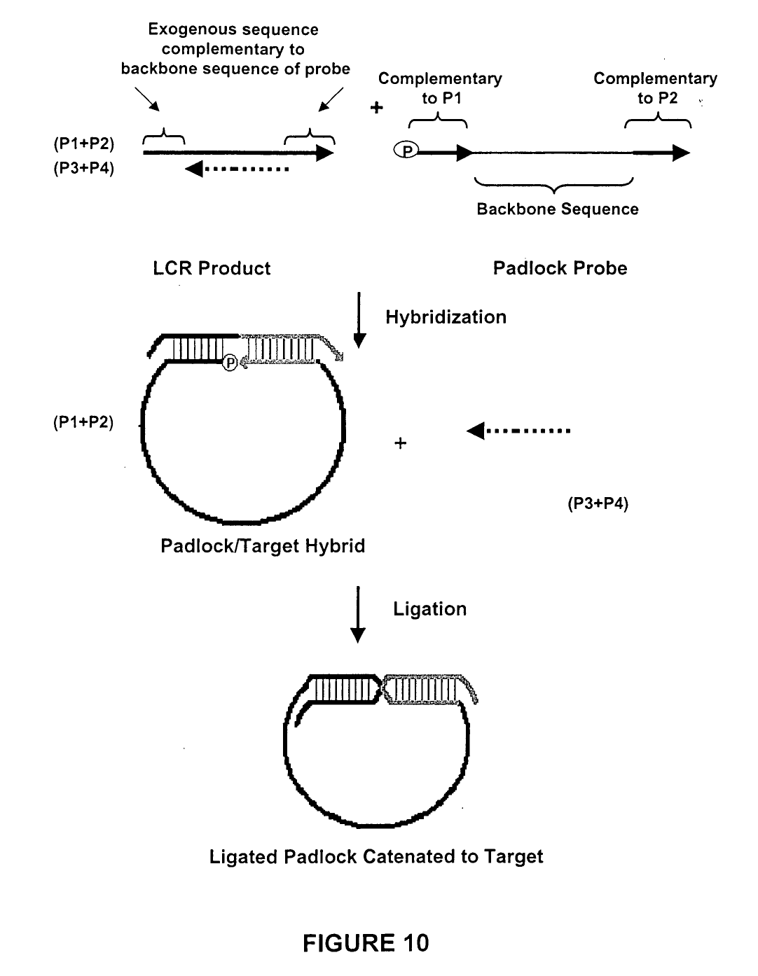

In the example shown in FIG. 9, primers P1 and P2 are longer than P3 and P4, where the LCR primers complementary to the target strand have additional nucleotides at the 5′ (P1) and 3′ (P2) ends that are not complementary to sequence on the target strand template. FIG. 10 illustrates an embodiment in which LCR primers for the target strand (P1 and P2) have additional nucleotides that are complementary to the backbone of a padlock probe for RCA. Preferably, the primers have 5 additional nucleotides. Additional complementary nucleotides permit the padlock probe to outcompete the P3+P4 LCR product for the P1+P2 template during hybridization and ligation of the padlock probe. After ligation, the circularized padlock probe will be wound around the target P1+P2 LCR product containing the target nucleotide sequence, topologically connecting the padlock probe to the target through catenation. Padlock formation can use the same thermostable ligase as that used for LCR, such that once the desired number of LCR cycles is complete, the padlock probe can be added directly to the reaction mixture. There is no need to set up a new reaction to carry out RCA after LCR, whereas a new reaction must be set up to carry out PCR amplification following LCR. In the reaction mixture, the padlock probe is allowed to hybridize to the desired template (e.g., the P1+P2 LCR product in FIG. 10) and the ends of the padlock probe are ligated. The process of padlock probe hybridization and ligation is allowed to proceed for a number of cycles to produce the desired number of ligated padlocks. RCA can then occur.

LCR provides advantages that can make it the method of choice for amplification of target nucleotide sequences, depending on circumstances. By way of example, an LCR product may be of shorter length than a PCR product. RCA of an LCR product produces a single-stranded amplification product whereas the double-stranded PCR product is denatured before it is used for hybridization. Advantageously, LCR may be used to generate a single-stranded product of defined sequence and length without the need for post-amplification trimming steps.

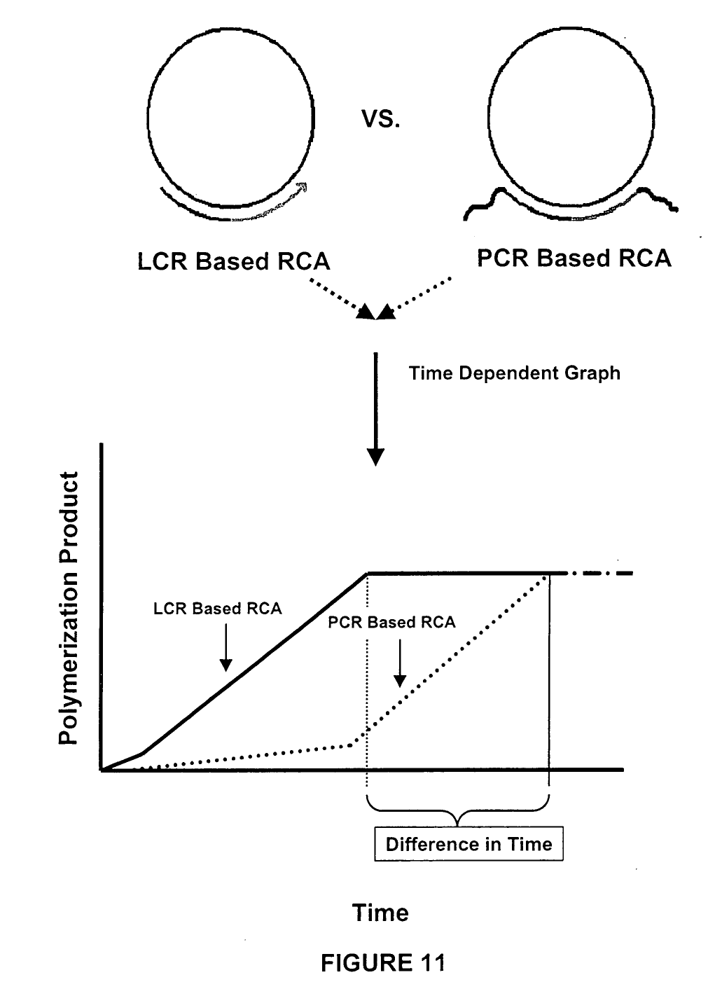

When an LCR product is used with an RCA probe, such as a padlock probe, to carry out linear amplification by RCA, the polymerase for RCA will not require hybridization of an additional oligonucleotide for polymerization. As illustrated in FIG. 11.A., LCR-based RCA requires only the LCR product and padlock probe for RCA to proceed. In LCR-based RCA, the LCR product is completely complementary to the padlock probe and thus it can function as a polymerization oligonucleotide when the isothermal polymerase is added to the reaction mixture. In LCR-based RCA, synthesis of single-stranded tandem repeats of the target nucleotide sequence is initiated using the circularized padlock probe as the template and the LCR product as the polymerization initiation oligonucleotide. In contrast, as illustrated in FIG. 11.B., PCR-based RCA requires the additional step of adding and hybridizing a polymerase oligonucleotide (shown as P5) to the padlock probe hybridized to the template. FIG. 11.C. illustrates the kinetics of LCR-based RCA and PCR-based RCA, demonstrating that LCR-based RCA avoids the lag phase associated with PCR-based RCA. Single-stranded tandem repeats of target nucleotide sequence generated by LCR-based RCA can be processed as described herein to generate single-stranded DNA products having defined sequence and length that can be used as disclosed herein.

Preferably, single-stranded DNA products having defined sequence and length generated in accordance with the methods described herein can be used for hybridization based diagnostics. Uses of such DNA products include identification of an organism or individual, or detection of SNPs in a patient sample. Advantageously, the methods described herein permit amplification of DNA or RNA from patient samples to generate single stranded DNA products in a relatively short amount of time, resulting in highly specific amplification of a target nucleotide sequence from a gene of interest from a patient sample using a highly streamlined protocol. In particular, LCR-based methods allow further streamlining of protocols, permit amplification without PCR, and can be used as a pre-amplification step to enrich the population of target nucleotide sequence in a mixture.

Advantageously, LCR-based RCA improves the efficiency of generating single stranded DNA product using RCA, especially when genomic DNA is used as a template. Typically, a problem with carrying out RCA using genomic DNA is that the ligated padlock probe is catenated to the genomic target and the RCA reaction is greatly hindered unless there is a free 3′ end nearby (Baner et al., 1998, Nuc Acids Res 26:5073-5078). It is possible to use PCR amplification of template containing the target nucleotide sequence, using PCR primers designed to introduce exogenous sequences that permit the padlock probe to hybridize near the free 3′ end of the PCR product (PCR-based RCA). However, the strand displacement properties of the isothermal polymerase used in RCA may cause the padlock probe to eventually “fall off” the template from the 3′ end to allow linear amplification to proceed. PCR amplification may be time consuming and in addition, the PCR product generated thereby may be longer than is deemed optimal for RCA. Having longer template sequence to which the padlock probe is catenated results in a longer lag phase before efficient RCA can proceed. RCA is not efficient until the isothermal polymerase displaces the circularized padlock from its complementary template. Thus, the reaction does not enter linear amplification until the template is displaced (FIG. 11.C.). Although this lag phase can be prevented or minimized by digesting the template prior to RCA, such an approach adds even more steps and time to the protocol. In contrast, LCR-based RCA as described herein allows amplification without PCR, saving time and materials. The shorter LCR products prevent the lag phase associated with template displacement from the 3′ terminus and no digestion of the padlock probe target sequence is needed after ligation of the probe (FIG. 11.C.). LCR-based RCA likewise does not require hybridization of an additional oligonucleotide for initiation of polymerization.

Although amplification of LCR products by RCA has been exemplified above and throughout the specification with reference to padlock probes, it will be appreciated that other RCA probes, such as preformed circular probes, can be used in place of padlock probes as a template for RCA.

Chip Applications

Amplification methods described herein can be performed entirely in solution, or alternatively, they may be performed either partially or entirely on a solid surface. In some embodiments of the present invention, nucleic acid amplification can be combined with analyses which utilize gene chips. For example, one or more primers used in LCR can be attached directly to a gene chip by methods well known in the art. In such embodiments, the LCR reaction occurs on the chip. In other embodiments, a capture probe which comprises a nucleotide sequence complementary to at least a portion of the LCR product can used to couple a completed LCR product to the chip.

By “capture probe” is meant a probe that is attached to a surface or another molecule and which comprises a sequence that is complementary to at least a portion of a sequence that is present in a nucleic acid of interest.

As used herein, “at least a portion” means a part of a nucleotide sequence that ranges between 5 bases and 10,000 bases in length. In some embodiments, the length is 5 bases, 6 bases, 7 bases, 8 bases, 9 bases, 10 bases, 11 bases, 12 bases, 13 bases, 14 bases, 15 bases, 16 bases, 17 bases, 18 bases, 19 bases, 20 bases, 21 bases, 22 bases, 23 bases, 24 bases, 25 bases, 26 bases, 27 bases, 28 bases, 29 bases, 30 bases, 31 bases, 32 bases, 33 bases, 34 bases, 35 bases, 36 bases, 37 bases, 38 bases, 39 bases, 40 bases, 41 bases, 42 bases, 43 bases, 44 bases, 45 bases, 46 bases, 47 bases, 48 bases, 49 bases, 50 bases, 51 bases, 52 bases, 53 bases, 54 bases, 55 bases, 56 bases, 57 bases, 58 bases, 59 bases, 60 bases, 61 bases, 62 bases, 63 bases, 64 bases, 65 bases, 66 bases, 67 bases, 68 bases, 69 bases, 70 bases, 71 bases, 72 bases, 73 bases, 74 bases, 75 bases, 76 bases, 77 bases, 78 bases, 79 bases, 80 bases, 81 bases, 82 bases, 83 bases, 84 bases, 85 bases, 86 bases, 87 bases, 88 bases, 89 bases, 90 bases, 91 bases, 92 bases, 93 bases, 94 bases, 95 bases, 96 bases, 97 bases, 98 bases, 99 bases, 100 bases, 110 bases, 120 bases, 130 bases, 140 bases, 150 bases, 160 bases, 170 bases, 180 bases, 190 bases, 200 bases, 250 bases, 300 bases, 350 bases, 400 bases, 450 bases, 500 bases, 600 bases, 700 bases, 800 bases, 900 bases, 1000 bases, 2000 bases, 3000 bases, 4000 bases, 5000 bases, 6000 bases, 7000 bases, 8000 bases, 9000 bases or 10,000 bases.

Base pairing interactions between the capture probe and a nucleic acid sequence of interest are used to facilitate coupling (capture) of a nucleic acid sequence of interest to the surface to which the capture probe is attached. In some embodiments of the present invention, an exogenous sequence present in one or more LCR primers can be used to couple the desired LCR primers to the chip. In some embodiments, the amplified sequences may be attached to a chip by hybridization between a capture probe and an exogenous sequence present in the single-stranded RCA amplification product. In this case, an exogenous sequence, which is identical to at least a portion of the capture probe sequence, is present in the RCA probe. Copying the RCA probe, generates an RCA amplification product having a plurality of exogenous sequences that are each complementary to a nucleotide sequence present in the capture probe. Base pairing interactions between the exogenous sequence present in the single-strand RCA amplification product and the capture probe facilitate attachment to the chip surface via the capture probe.

In some embodiments of the present invention, a capture probe is attached to a detector. In such embodiments, the capture probe can be used to determine whether a nucleic acid comprising a sequence complementary to at least a portion of the capture probe sequence is present in a sample. In certain examples, a capture probe can be coupled directly to a detector or otherwise coupled to a detection zone that is present on a surface.

In other embodiments of the present invention, a plurality of capture probes are attached to a gene chip to form an array. An array of capture probes as disclosed herein provides a medium for detecting the presence of targets in a sample based on rules for nucleic acid hybridization. Generally, an array of capture probes refers to an array of probes immobilized to a support, where the sequence (the identity) of each capture probe at each location is known. Alternatively, an array of capture probes may refer to a set of capture probes that are not immobilized and can be moved on a surface, or may refer to a set of probes coupled to one or more particles such as beads. In some embodiments, the process of detecting target nucleic acids hybridized to capture probes is automated. Microarrays having a large of number of immobilized capture probes of known identity can be used for massively parallel gene expression and gene discovery studies. A variety of detection methods for measuring hybridization of detector reagents are known in the art, including fluorescent, colorimetric, radiometric, electrical, or electrochemical means.

Diverse methods of making oligonucleotide arrays are known, for example as disclosed in U.S. Pat. Nos. 5,412,087, 5,143,854, and 5,384,261 (the disclosures of which are incorporated herein by reference in their entireties) and accordingly no attempt is made to describe or catalogue all known methods. In some embodiments of the present invention, arrays of capture probes are set out on the surface of a chip. Some chips can contain an array of gene-specific capture probes for hybridization with select nucleic acid sequences, such as target sequence LCR products. Universal chips can contain an array of capture probes that comprise one or more sequences for use in capturing nucleic acids that comprise one or more exogenous sequences that are added to the LCR primers and/or the RCA probe.

One aspect of the present invention provides a universal chip having capture probes attached to a support that functions as an electrical contact surface or electrode to detect nucleic acid hybridization. Methods for attaching oligonucleotides to an electrical contact surface are well known, for example as disclosed in any of U.S. Pat. Nos. 5,312,527, 5,776,672, 5,972,692, 6,200,761, and 6,221,586, the disclosures of which are incorporated herein by reference in their entireties.

In the fabrication process, many other alternative materials and processes can be used. The substrate may be glass or other ceramic material; the bottom silicon dioxide can be replaced by silicon nitride, silicon dioxide deposited by other means, or other polymer materials; the conducting layer can be any appropriate material such as platinum, palladium, rhodium, a carbon composition, an oxide, or a semiconductor. For amperometric measurement either a three-electrode system consisting of a working electrode, counter electrode and reference electrode or a two-electrode system comprising a working and a counter/reference electrode is necessary to facilitate the measurement. The working electrodes should provide a consistent surface, reproducible response from the redox species of interest, and a low background current over the potential range required for the measurement. The working electrodes may be any suitable conductive materials, preferably noble metals such as gold and platinum, or conductive carbon materials in various forms including graphite, glassy carbon and carbon paste. For a three electrode system the reference electrode is usually silver or silver/silver chloride, and the counter electrode may be prepared by any suitable materials such as noble metals, other metals such as copper and zinc, metal oxides or carbon compositions. Alternatively, the conducting layer can be prepared by screen printing of the electrode materials onto the substrate. Screen printing typically involves preparation of an organic slurry or inorganic slurry of an electrode material, such as a fine powder of carbon or gold, onto the substrate through a silk screen. The electrode material slurry may be fixed on the surface by heating or by air drying. The electrode may be any suitable conductive material such as gold, carbon, platinum, palladium, indium-tin-oxide. It is often advantageous to coat the electrode surface with a material such as avidin, streptavidin, neutravidin, or other polymers, to increase the immobilization of capture probes. Methods for the attachment include passive adsorption and covalent attachment.

If gold is chosen for the conducting layer, the layer can be evaporated, sputtered, or electroplated. A low temperature oxide layer can be replaced by spin-on dielectric materials or other polymer materials such as polyimide, or parylene. Reagent and electrical connections can be on the same side of a chip or on adjacent sides, though the opposite side configuration is preferred. Materials, temperatures, times, and dimensions may be altered to produce detectors, preferably chips, having substantially the same properties and functionality, as will be appreciated by those of skill in the art. Materials, temperatures, times, and dimensions may be altered by one of skill in the art to produce chips having the properties desired for any particular embodiment.

As related to some aspects of the present invention, the capture probes are immobilized on a support having an array of electrodes sandwiched between two layers of silicon dioxide insulator attached to the silicon substrate, where a supporting layer is opposite the silicon substrate and the chip is oriented such that the silicon substrate is on the top and the supporting layer is on the bottom, as disclosed in U.S. patent application Ser. No. 10/121,240, METHOD FOR MAKING A MOLECULARLY SMOOTH SURFACE, filed Ap. 10, 2002, the disclosure of which is incorporated herein by reference in its entirety. In some embodiments, gold electrodes are used. Alternatively, carbon electrodes such as graphite, glassy carbon, and carbon paste can be used. In this embodiment, access to the surfaces of the working electrodes, where the capture probes are immobilized, is through windows through the silicon substrate and top layer of insulator on the top surface of the chip. Windows on the underside (etched through the supporting layer and the bottom layer of insulator) allow access to a counter (or detector) electrode and a reference electrode. For gold electrodes, the two types of electrodes in the chip are selectively interconnected by deposited gold wiring within the insulating layer or by other methods known in the art. Access to the working electrode, reference electrode, and counter electrode allows a complete circuit to be formed which will enable standard techniques in the art, such as amperometric measurements, to be performed using the chip. An electrode potential applied to the working electrode, where the electrochemically active materials are present through association with the capture probe and complementary exogenous sequences, will produce current proportional to the amount of exogenous sequence attached to the capture probes.

Detection of Amplified Nucleic Acids

Nucleic acids that are amplified using the methods described herein can detected by a number of different methods. In some embodiments, the amplified nucleic acids are bound to a chip then detected electrochemically. In other embodiments, a detection probe is used to facilitate the binding of one or more detector molecules. As used herein, a “detection probe” is a nucleic acid probe which comprises a nucleotide sequence that is complementary to at least a portion of a nucleotide sequence in the single-stranded RCA amplification product and which directly or indirectly provides a signal indicative of the presence of a target nucleotide sequence. In preferred embodiments, a detection probe is capable of binding to an exogenous sequence that is present in the single-stranded RCA amplification product. In certain embodiment of the present invention, a detection probe can also comprise a detector molecule. Examples of detector molecules include, but are not limited to, ligands for antibodies, lectins and other binding molecules. Additionally, a detector molecule can be a charged or uncharged molecule which binds to DNA or a molecule which actively generates a signal (signal generating molecule). A detection probe having a signal generating molecule coupled thereto may also referred to as a signal generating probe. A detector molecule can, but need not, be used in connection with a detection probe.

All of the above-described probes and molecules can be referred to generally as “detector reagents.” In some embodiments of the present invention, the detector reagent can be coupled to the amplified nucleic acid by direct binding. An example of a direct binding detector reagent is ruthenium. In other embodiments of the present invention, the detector reagents can be coupled to the amplified nucleic acid indirectly. An example of an indirectly coupled detector reagent is a redox enzyme/secondary antibody conjugate, which is bound to a primary antibody, which in turn is bound to fluorescein that is present in a detection probe. In this example, the detection probe is bound to the amplified nucleic acid sequence via hydrogen bonding. Additionally, in some embodiments, a plurality of detector reagents function together to produce a detectable signal. In still other embodiments, a plurality of detector reagents are used separately to produce a plurality of separate detectable signals.

The detection methods described herein are useful for detecting amplified nucleic acids in solution as well as amplified nucleic acids that are attached to a solid support, such as a gene chip. In some embodiments, a target nucleotide sequence of interest is amplified in solution and then detected after attachment to a solid support. In other embodiments, the target sequence to be detected is directly amplified on the solid support. In preferred embodiments, the solid support is a gene chip.

The following is general procedure for detecting a nucleic acid sequence amplified on a gene chip. A first LCR primer comprising a nucleotide sequence complementary to a portion of a target nucleotide sequence of interest is attached to a gene chip. A second LCR primer comprising a nucleotide sequence complementary to the remainder of the target sequence is incubated together with the first primer and the target sequence. LCR is performed and unbound target and primers are removed from the chip by washing. An RCA probe comprising both a sequence complementary to at least a portion of the LCR product and an exogenous sequence identical to at least a portion of a detection probe sequence is incubated with the attached LCR product. Polymerization is then initiated from the free 3′ end of the LCR product using a processive DNA polymerase. Preferably, the target nucleotide sequence from the sample is elongated by approximately 10 to 10,000 copies of the RCA probe. After the amplification step, the RCA probe is removed to produce a single-stranded RCA product which is attached to the chip and which comprises a plurality of alternating exogenous and target sequences. The amplification product is then incubated with a detection probe having a detector molecule attached thereto. Finally, the detector molecule is detected using a technique that is appropriate for the particular detector molecule that is used.

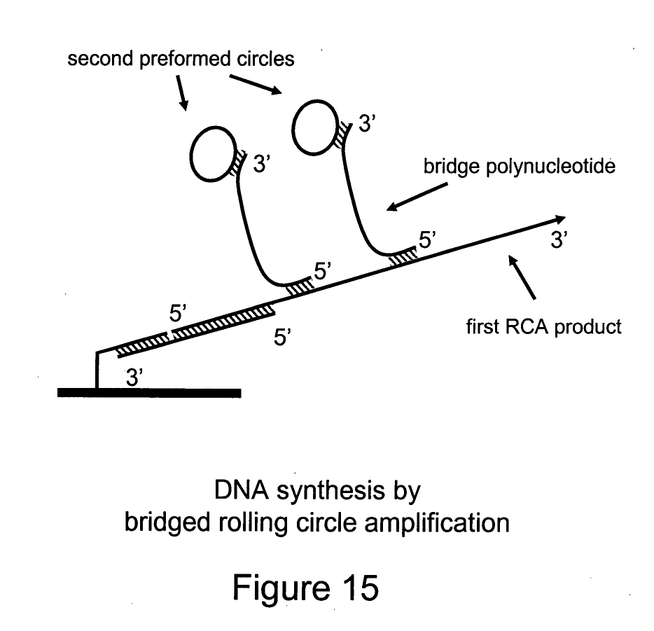

The general amplification technique described above can be modified so as to increase the number of binding sites for detector probes, thereby increasing the intensity of the detectable signal. One technique is known as “bridge” amplification or “branch” amplification. Bridge amplification is illustrated in FIG. 15. In one example of bridge amplification, LCR is used to preamplify a desired target sequence. One of the LCR primers used for this step comprises an exogenous nucleotide sequence complementary to at least a portion of a capture probe sequence. After the preamplification, the LCR products are attached to a gene chip via hybridization between the capture probe and an the exogenous nucleotide sequence. Next, a first RCA probe which comprises a nucleotide sequence complementary to at least a portion of the LCR product, a first exogenous sequence identical to at least a portion of a detection probe sequence, and a second exogenous sequence identical to at least a portion of a sequence in a bridge nucleic acid, is incubated with the LCR product. In some embodiments, the RCA probe is a padlock probe. In other embodiments, the RCA probe is a preformed circle. Extension of the 3′-end the LCR product generates a first single-stranded RCA product comprising repeated copies of target sequence interspersed with the nucleotide sequences complementary to the first and second exogenous sequences contained in the RCA probe. Following extension, a bridge nucleic acid, which comprises a nucleotide sequence complementary to at least a portion of the second exogenous sequence in the first RCA product and which comprises a nucleotide sequence complementary to a nucleotide sequence contained in a second RCA probe, is hybridized to the first RCA product. The second RCA probe is then hybridized to the free end of the bridge sequence and a second RCA procedure is performed, thereby producing a second RCA product.

The second RCA probe that is used in branch amplification can, but need not, comprise one or more exogenous sequences. In some embodiments, the second RCA probe comprises a first exogenous sequence identical to at least a portion of a detection probe sequence. In other embodiments, the second RCA probe does not comprise an exogenous sequence identical to at least a portion of a detection probe sequence.

Subsequent to the amplification of the second RCA product, a detection probe having a detector molecule attached thereto is hybridized to the complementary first exogenous nucleotide sequence acid in the combined amplified product (for example, the first RCA product and the second RCA product). The detector molecule is then detected using a technique that is appropriate for the particular detector molecule that is attached to the detection probe.

Although illustrated here with exogenous sequences, it will be appreciated that the nucleotide sequence in the bridge nucleic acid that is complementary to the first RCA product may be complementary to any portion of the first RCA product. For example, this bridge sequence can be complementary to the entire target nucleotide sequence or a portion thereof. Alternatively, as illustrated above, the nucleotide sequence in the bridge nucleic acid that is complementary to the first RCA product can be complementary to all or a portion of an exogenous sequence contained in the first RCA product. In some embodiments of the present invention, the bridge nucleic acid also comprises one or more exogenous sequences. In certain embodiments, the bridge sequence can include a first exogenous sequence that is complementary to at least a portion of a detection probe.

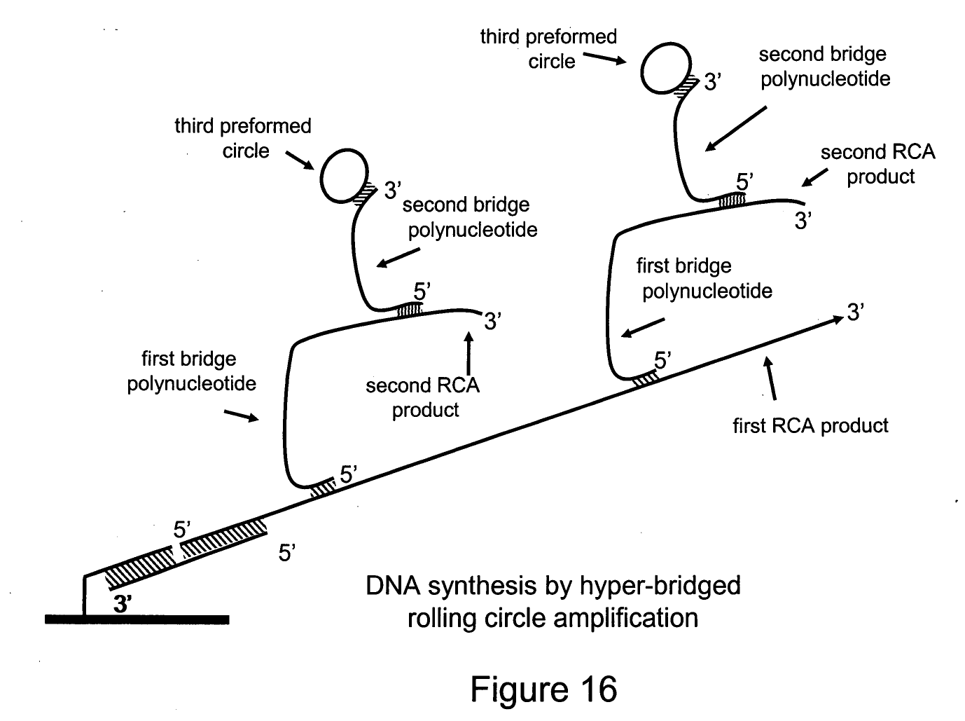

Further, when a bridge amplification technique is used, it can be advantageous to increase the extent of branching using a technique known as “hyperbridging” or “hyperbranching.” An example of hyperbridging is shown in FIG. 16. Here, a second bridge nucleic acid comprising a sequence complementary to a sequence in the second RCA product and a sequence complementary to a sequence in a third RCA probe is provided. The second bridge nucleic acid is hybridized to the second RCA product and a third RCA procedure is performed with the third RCA probe, thereby producing a third RCA product. As indicated above, the third RCA probe and the second bridge nucleic acids can contain one or more exogenous nucleotide sequences. In some embodiments, the exogenous nucleotide sequence is a first exogenous nucleotide sequence that is complementary to at least a portion of a detection probe.

Subsequent to the amplification of the third RCA product, a detection probe having a detector molecule attached thereto is hybridized to the complementary first exogenous nucleotide sequence acid in the combined amplified product (for example, the first RCA product, the second RCA product and the third RCA product). The detector molecule is then detected using a technique that is appropriate for the particular detector molecule that is attached to the detection probe.

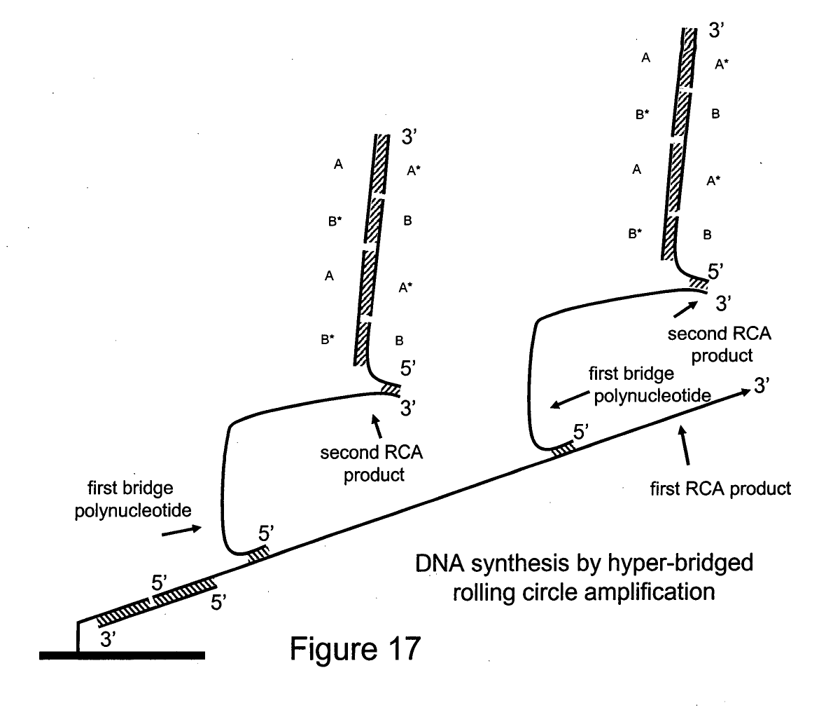

Some embodiments of the present invention also contemplate elongation of any of the above-described single-stranded RCA products using methods that increase the length of available nucleic acid for detector reagent binding without necessarily replicating the target sequence. One such method is known as head-to-tail polymerization. In head-to-tail polymerization, which is depicted in FIG. 17, exogenous nucleotide sequences present in the single-stranded RCA product are targeted. In certain embodiments of the present invention, in which the RCA product is attached to a chip, head-to-tail polymerization is useful for additional building up the amount of DNA physically present on the electrodes. Typically, three different oligonucleotides will be used as shown here: the first oligomer is complementary to a exogenous nucleotide sequence of the hybridized target strand, and contains a sequence A at its 5′ end; the second oligomer has a sequence 5′-A*B-3′, where A* is complementary to A; the third oligonucleotide has sequence 5′-AB*-3′. As depicted in FIG. 17, these oligomers can form a polymeric product as shown. The head-to-tail polymerization can continue until the strand reaches a desired length. Generally, when performing head-to-tail polymerization, the ultimate length of the polynucleotide is limited in part by a competing cyclization reaction of the head-to-tail oligomers. A higher concentration of head-to-tail oligomers in the liquid medium will generally produce longer linear polymers attached to the electrode, however.

It is generally advantageous to use an elongation technique such as rolling circle amplification or head-to-tail polymerization in conjunction with a hyperbridging process. Elongation generally serves the purpose of adding nucleic acid material that can be detected electrochemically to help distinguish successfully amplified nucleic acids from those that have not been amplified, but the process can also be beneficial since longer nucleic acids provide more locations in which additional detector reagents and/or bridges for further amplification can be attached.

In addition, bridging and hyperbridging can be particularly useful techniques since the amount of nucleic acid present can be increased exponentially. Additional discussion of bridging and hyperbridging techniques can be found, for example, in: Urdea, Biotechnology 12:926 (1994); Horn et al., Nucleic Acids Res. 25(23):4835-4841 (1997); Lizardi et al., Nature Genetics 19, 225-232 (1998); Kingsmore et al. U.S. Pat. No. 6,291,187); Lizardi et al. (PCT application WO 97/19193); all of which are hereby incorporated by reference.

Other methods for detecting nucleic acids can be used in connection with a single-stranded RCA product with or without use of a bridging or further amplification step. Assuming that a detection probe is used to associate a redox enzyme with hybridized nucleic acid, the following two examples outline steps that could be taken in an assay that does not feature bridging, and an assay that does feature bridging respectively.

A method without bridging generally includes the following steps: (1) perform LCR reaction (may be used to determine specificity through ligation as previously described); (2) bind the RCA probe to the LCR product; (3) perform RCA using the bound LCR product to initiate extension; (4) add a detection probe containing an epitope, such as fluorescein; (5) add an antibody linked to a redox moiety such that the antibody is capable of binding to the epitope; and (6) detect the redox activity related to the presence of the redox moiety.

A method that features a bridging step generally includes the following: (1) perform LCR reaction (may be used to determine specificity through ligation as previously described); (2) bind the RCA probe to the LCR product; (3) perform RCA using the bound LCR product to initiate extension; (4) add a bridge nucleic acid and a second RCA probe; (5) perform RCA at the bridge; (6) add a detection probe containing an epitope, such as fluorescein, wherein the detection probe hybridizes to the single-stranded RCA products; (7) add an antibody linked to a redox moiety such that the antibody is capable of binding to the epitope; and (8) detect the redox activity related to the presence of the redox moiety.

After performing the amplification reaction, the increased amount of nucleic acid is used to generate a larger and more detectable signal. This can be advantageous for assay purposes since both capture probe and/or the unamplified target sequence typically produce some detectable signal. In embodiments in which on chip amplification is performed, the additional nucleic acid further increases contrast between amplified and unamplified capture probes.

It will be appreciated that in any of the embodiments described herein that nucleic acid analogs can be used to increase the stability of probes and primers. For example, nucleic acid analogs such as methyl phosphonates and PNAs can be used as capture probes. In other embodiments, nucleic acid analogs such as methyl phosphonates and PNAs can be used as detection probes.

In addition to the above, amplification techniques described herein can be used alone or in conjunction with other signal enhancing techniques such as catalytic detection. Some embodiments include the steps of: (1) hybridizing an exogenous sequence of an LCR product to a capture probe immobilized on an electrode surface; (2) performing rolling circle amplification using a polymerase with high processivity and strand displacement capability; (3) binding a detection probe to the amplified product on the chip; and (4) detecting catalytic signal generated from a highly electrochemically reactive enzyme.

In some embodiments of the present invention, horseradish peroxidase (HRP) is used in generate electrons by the redox conversion of hydrogen peroxide to water. HRP is particularly useful as a detector reagent because of its stability, high turn over rate, and the availability of sensitive electrochemical mediators. Other enzymes such as phosphatases, other peroxidases including microperoxidase, and oxidases can also be used for this purpose. In some embodiments, the electrochemical detection using HRP proceeds as follows:
HRP_{R}+H₂O₂+2H⁺→HRP_{O}+2H₂O
HRP_{O}+Med_{R}→HRP_{R}+Med_{O}
Med_{O}+ne⁻→Med_{R}
wherein Med denotes an electron transfer mediator that shuttles electrons between the enzyme and the electrode surface. Useful mediators include tetramethylbenzidine (TMB) and ferrocene derivatives. It will be appreciated by those of skill in the art that other enzymes, particularly other peroxidases can be used instead of HRP, and that other electron transfer mediators can be used instead of TMB. Further examples of HRP and other redox mechanisms are described in U.S. Provisional Patent Application No. 60/518,816, entitled NUCLEIC ACID DETECTION METHOD HAVING INCREASED SENSITIVITY, filed on Nov. 10, 2003, the disclosure of which is incorporated herein by reference in its entirety. A non-exhaustive description of some of the available detection methods is set out below, however, it will be appreciated that any methods of nucleic acid detection that are known in the art can be used with the methods described herein.

The redox enzyme and other detection methods described herein have substantial utility in embodiments in which amplified nucleic acids are bound to an electrochemical detection zone on a gene chip. In an exemplary embodiment of the redox system, a chip-bound amplified nucleic acid is hybridized with a detection probe having one or more detector molecules included thereon. An antibody then binds specifically to the detector molecules and the signal is amplified using enzyme-linked secondary antibody. If the secondary antibody is conjugated to a redox enzyme, such as HRP, the signal can be detected using amperometry.

In addition to the above-described methods, numerous methods, which do not utilize detection probes, are available for detecting amplified nucleic acids. In some embodiments of the present invention, nucleic acid hybridization can be detected using a transition metal complex capable of oxidizing at least one oxidizable base in an oxidation-reduction reaction under conditions that cause an oxidation-reduction reaction between the transition metal complex and the oxidizable base, where the amplified target nucleotide sequence contains at least one oxidizable base. The oxidation-reduction reaction indicating hybridization is detected by measuring electron transfer from each oxidized base, as disclosed in U.S. Pat. No. 5,871,981, the disclosure of which is incorporated herein by reference in its entirety.

In another embodiment, amplified nucleic acids bound by capture probes that are immobilized on gold or other electrodes may be carried out using methods disclosed by Steele et al. (1998, Anal. Chem 70:4670-4677), the disclosure of which is incorporated herein by reference in its entirety. For example, multivalent ions with 2, 3, or 4 positive charges are used, which are capable of electrochemical detection by direct reaction without affecting the nucleic acid. In some embodiments these ions bind electrostatically to nucleic acid phosphate irrespective of whether it is in the double-helical or single-stranded form. The presence or absence of an amplified hybridized sequence is determined for each capture probe, based on electron transfer measurements taken at each capture probe site. By way of example, this direct electrochemical detection method can be used to detect the presence of a certain allele in a biological sample. A specific allele may be detected in a sample containing a target sequence by using LCR as described previously herein. The result of such procedure is that an LCR product will only be formed using the correct allele-specific primer set. Since one primer of each allele-specific primer set will contain an exogenous nucleotide sequence for attachment to a capture probe, both single primers and complete LCR products will bind to capture probe. However, if a padlock probe is used or if hybridization stringency is controlled, RCA amplification will only occur for complete LCR products. Thus, only primer pairs that can produce an LCR product upon binding to the target sequence will be amplified on the chip. All other primer primers will be unamplified. Suitable transition metal complexes that bind nucleic acids electrostatically can then be used to generate a detection current that is proportional to the length of the nucleic acids bound to the chip. Transition metal complexes whose reduction or oxidation is electrochemically detectable in an appropriate voltage regime include Ru(NH₃)₆³⁺, Ru(NH₃)₅pyridine³⁺ and other transition metal complexes that can be determined by one of skill in the art.

According to one embodiment of the present invention, oligonucleotide capture probe sequences may be designed to be redox inactive, or to have very low redox activity, for example as disclosed in U.S. Pat. No. 5,871,918, the disclosure of which is incorporated herein by reference in its entirety.

The occurrence of the oxidation-reduction reaction may be detected according to any suitable means known to those skilled in the art. For example, the oxidation-reduction reaction may be detected using a detection electrode to observe a change in the electronic signal which is indicative of the occurrence of the oxidation-reduction reaction. Suitable reference electrodes will also be known in the art and include, for example, silver, silver/silver chloride electrodes. The electronic signal associated with the oxidation-reduction reaction permits the determination of the presence or absence of hybridized tags by measuring the Faradaic current or total charge associated with the occurrence of the oxidation-reduction reaction. The current depends on the presence of the positively charged redox ion closely associated with the electrode, which in turn depends on the amount of nucleic acid phosphate hybridized to the electrode. The electronic signal may be characteristic of any electrochemical method, including cyclic voltammetry, normal pulse voltammetry, differential pulse voltammetry, chronoamperometry, and square-wave voltammetry. The amount of nucleic acid hybridized to a capture probe is determined by subtracting the current or total charge characteristic of the probes and other molecules bound to the electrode in the starting state from the current or total charge measured after the hybridization/amplification reaction.

Additional methods for the amplification and detection of target nucleotide sequences have been described in U.S. Provisional Patent Application No. 60/488,177, entitled INVASIVE CLEAVAGE REACTION WITH ELECTROCHEMICAL READOUT, filed Jul. 16, 2003; U.S. Provisional Patent Application No. 60/497,821, entitled OLIGONUCLEOTIDE SEQUESTERING AGENTS AND METHODS OF USE, filed Aug. 25, 2003; and U.S. Provisional Patent Application No. 60/519,568, entitled NUCLEIC ACID HYBRIDIZATION METHODS, filed Nov. 12, 2003, the disclosures of which are incorporated herein by reference in their entireties.

Kits

Some aspects of the present invention also contemplate a kit for amplifying and detecting a target nucleic acid. Some embodiments of the kits contemplated herein comprise a detector reagent. In some embodiments, the detector reagent is an electrochemical detection reagent. In other embodiments, the detector reagent is a signal generating molecule such as a redox enzyme conjugated to an antibody. In still other embodiments, the detector reagent is a detection probe coupled to a detection molecule.

Some embodiments of kits contemplated herein comprise a chip having a plurality of capture probes attached thereto. In some embodiments, the chips can comprise an array of capture probes. In other embodiments, the capture probes each comprise one or more sequences complementary to an specific exogenous sequence. Arrays of capture probes comprising sequences complementary to exogenous sequences of different length and nucleotide sequence are also contemplated. Such kits are useful for multiplexed DNA analysis.

Still other embodiments of kits also include LCR primers that are complementary to at least a portion of a desired target sequence. In such embodiments, one or more RCA probes having sequence(s) complementary to at least a portion of the sequence which is formed by template-specific ligation of the included LCR primers can be included with the kit. LCR primers and/or the RCA probe can comprise one or more exogenous sequences which facilitate enzymatic processing, hybridization with a capture probe and/or hybridization with a detection probe.

In some embodiments of the present invention, the kits contemplated herein also optionally include instructions for using the reagents provided with the kit.

Uses of Single-Stranded DNA Molecule Generation Methods

In accordance with another aspect of the present invention, methods for generating single-stranded DNA molecules of defined sequence and length from template containing a target nucleotide sequence as described herein, may be used to identify an organism or individual. A sample including template is obtained from an organism or individual, or from a multiplicity of organisms or individuals, where the template contains at least one target nucleotide sequence, and the template may be genomic DNA, cDNA, or RNA. Template is amplified using one or more specially designed primers or probes, conversion of double-stranded amplification products into single-stranded amplification products is carried out if necessary, and single-stranded amplification products are trimmed as described herein to yield the desired set of DNA molecules of defined sequence and length, in accordance with the methods of the present invention as described herein. In one embodiment, the primers are chosen so that the sizes of the molecules in the set of single stranded DNA molecules are sufficient to identify a specific organism, where size may be measured as mass, nucleotide sequence, or length of the DNA molecule. In a preferred embodiment, template is amplified using specially designed primers or probes and double-stranded amplification products are produced, then the double-stranded amplification products are converted into single-stranded amplification products, and single-stranded amplification products are trimmed as described herein to yield the desired set of DNA molecules of defined sequence and length. In another preferred embodiment, template is amplified using specially designed primers or probes and single-stranded amplification products are produced, then the single-stranded amplification products are trimmed as described herein to yield the desired DNA molecule of defined sequence and length. The mass or nucleotide sequence of each single-stranded DNA molecule having the desired sequence and length can be determined, for example using mass spectroscopy to rapidly determine mass and/or nucleotide sequence, and the mass or nucleotide sequence can be used to identify an organism or an individual using tools available to one of skill in the art. In another embodiment, this method can be carried out using template from a multiplicity of organisms or individuals, the nucleotide sequence of each of a multiplicity of single-stranded amplification products is determined, and the masses or nucleotide sequences can be used to identify multiple organisms or individuals. In yet another embodiment, this method can be carried out using a sample from a multiplicity of organisms and individuals wherein the sample including template is obtained from a mixture of organisms or individuals, or alternately wherein multiple samples, each sample obtained from a single organism or individual, are pooled to create a single pooled sample for amplification, conversion, trimming, sequencing, and identification in accordance with the methods described herein.

### EXAMPLES

### Example 1

### Sample Preparation and Amplification

Materials.

Oligonucleotides were synthesized with phosphoramidites purchased from Glen Research. All enzymes were purchased from New England Biolabs Inc. (Beverly, Mass.), except for Taq DNA polymerase (Stratagene, La Jolla Calif.). Deoxyribonucleotide triphosphates (dNTP's) were also acquired from Stratagene. γ-³²P-ATP (3000 Ci/mmol) was obtained from Perkin Elmer Life Sciences (Boston, Mass.). Microquick spin columns were purchased from Roche Molecular Biochemicals (Indianapolis, Ind.). Oligonucleotides were synthesized using the phosporamidite method on an ABI 394 DNA synthesizer.

Preparation of Genomic DNA from Blood/Cell Lines or Tissue Samples.

Genomic DNA was prepared according to manufacturer's instructions using QuiAamp Blood DNA and QiAamp DNA kits (Quiagen, Valencia, Calif.). Similar kits are available for processing RNA.

Polymerase Chain Reaction.

PCR conditions were optimized for desired yield and specific template, quantity of genomic DNA, primers, and other components of the reaction, as well as the cycling conditions and specific temperatures. A specific illustration of optimized PCR conditions is found below. In certain conditions, it was necessary to inactivate components of the PCR reaction, for example by the use of phosphatase to inactivate dNTPs, or protease to inactivate DNA polymerase. (Werle et al., 1994, Nucleic Acids Res 22:4354-5)

### Example 2

### Generation of Single Strand DNA, Trimming, and Hybridization to Complementary Strand

Generation of Single Strand DNA by Lambda Exonuclease Digestion

The PCR reaction of Example 1 was supplemented to a final concentration of 50 μg/ml Bovine Serum Albumin (BSA) prior to removal of the targeted (5′ phosphorylated) DNA strand by lambda exonuclease according to the manufacturer's protocol, and recovery of the desired single-stranded DNA, if BSA was not added to the PCR reaction buffer. Concentration and incubation times varied, depending on yield from the PCR reaction (see specific example below). Heat inactivation of the enzyme for 10 minutes at 75° C. was desirable prior to subsequent steps.

Trimming of Single Stranded DNA to Desired Size.

Auxiliary oligonucleotides in a compatible buffer were provided to generate the double-stranded restriction endonuclease recognition site. The amount of enzyme and incubation conditions varied depending on amount of single-stranded product (see specific example below). To increase storage stability of final product at −20° C., heat inactivation for 20 minutes at 65° C. is recommended.

Hybridization to Complementary Strand.

The resulting single-stranded DNA was hybridized to its radioactive complementary strand for visualization after gel electrophoretic separation. Alternatively, it can be used for electrochemical SNP detection when allowed to hybridize to a test sequence immobilized to a solid support.

### Example 3

### Amplification of the Region of the S241→F of the p53 Tumor Suppressor Gene

Polymerase Chain Reaction

In this Example, polymerase chain reaction is used to produce a 55 bp pair product containing the S241→F SNP region of the p53 tumor suppressor gene which is then used to produce a specific 17 base single-stranded nucleic acid containing this SNP region. Amplification reactions were conducted using 25 ng of GDNA (from blood or cells) in a buffer containing 67 mM Glycine-KOH (pH 9.4), 2.5 mM MgCl₂, 50 μg/ml BSA, 0.625 Units Taq DNA polymerase, 0.2 mM dNTP's, and 0.2 μM of each primer, using the following protocol: 94° C. 2 minutes, followed by 30 cycles of 30 seconds at 94° C. 30 seconds at 64° C., and 30 seconds at 72° C. The following oligonucleotide primers were used:

| | |
|---|---|
| Primer 26.1′P: | |
| 5′ P-ATA GGA TGG TTC ATG CCG CCC ATG | (SEQ ID NO: 1) |
| CA 3′ | |
| Primer 27.2: | |
| 5′ TGG GGA TGA ACT ACA TGT GTA ACA | (SEQ ID NO: 2) |
| GTT 3′ | |

Lambda Exonuclease Digestion:

2.5 units lambda exonuclease were added to the PCR reaction, followed by incubation for 20 minutes at 37°. The enzyme was inactivated by incubation for 10 minutes at 75° C.

Fok I Digestion:

The digestion reaction contained 650 nM of each of two auxiliary oligonucleotides in 50 mM potassium acetate, 20 mM Tris-acetate, 10 mM magnesium acetate, 1 mM dithiothreitol, pH 7.9, added to the samples prior to incubation for 20 minutes at 37° C. with 4 units FokI. The enzyme was inactivated by incubation at 65° C. for 20 minutes. The auxiliary oligonucleotides were:

| | |
|---|---|
| (24.1′) | |
| 5′ TGT TAC ACA TGT AGT TCA TCC | (SEQ ID NO: 3) |
| CCA 3′ | |
| (26.1′) | |
| 5′ ATA GGA TGG TTC ATG CCG CCC | (SEQ ID NO: 4) |
| ATG CA 3′ | |

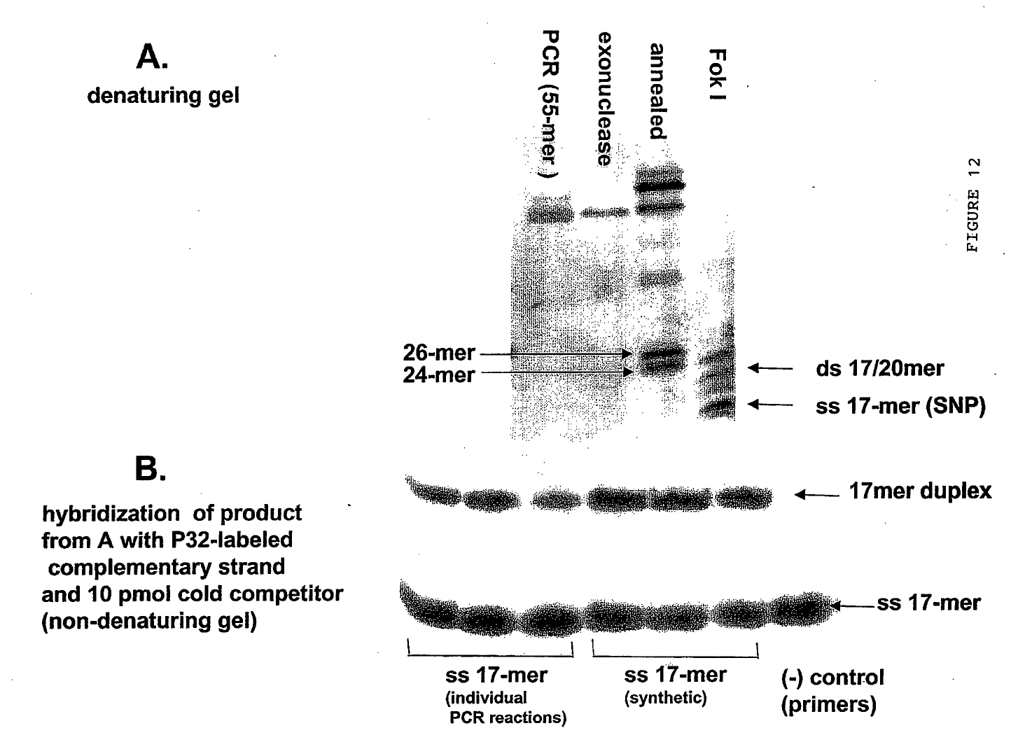

The products produced by PCR, exonuclease digestion, auxiliary oligonucleotide addition and FokI digestion were analyzed by electrophoresis on a denaturing polyacrylamide gel. FIG. 12A shows the 55 bp PCR product prior to conversion (Lane 1) as well as the expected single-stranded conversion product after processing with FokI (Lane 4). In particular, lane 4 includes a prominent band which corresponds to the expected 17 base single-stranded nucleic acid product which contains the sequence encoding the SNP (see FIG. 12B).

Hybridization to Complementary Strand:

The single-stranded 17-mer single-stranded DNA product was hybridized to the test sequence (17.1′) 5′ ³²P-ATG CAG GAA CTG TTA CA 3′ (SEQ ID NO: 5) by 15 minute incubation at room temperature. The test sequence 17.1′ (SEQ ID NO: 5) was phosphorylated by end-labeling, as follows: 0.5 μM oligonucleotide was incubated for one hour at 37° C. with 20 μCi of γ-³²P-ATP (3000 Ci/mmol) and 10 units T4 polynucleotide kinase in 70 mM Tris-HCl, pH 7.6, 10 mM MgCl₂, 5 mM dithiothreitol. To quantify the amount of product made, reactions were spiked with unphosphorylated test sequence 17.1′ and the fraction of the 17-mer duplex was estimated by comparing the intensity of excess ³²P single-stranded 17-mer to the total mixture (duplex+excess probe).

To determine visualize the binding of the single-stranded 17 base FokI digestion product to the test sequence 17.1, the above hybridization reactions were analyzed by electrophoresis on a non-denaturing polyacrylamide gel. FIG. 12B shows that the single-stranded 17 base nucleic acid obtained by FokI digestion hybridizes specifically to labeled test sequence 17.1 (see lanes 4-6, 17-mer duplex). Accordingly, the single-stranded 17 base nucleic acid generated by FokI digestion contains the expected SNP containing target sequence.

### Example 4

### Multiplexing, Detection of Three SNPs in the p53 Tumor Suppressor Gene

This experiment is analogous to Example 3 above, except that three (or more) sequences are amplified simultaneously, in the same tube. The primers are designed for length such that the optimum PCR temperatures are similar. For a mixture of SNPs constituting C176F, S241F and R248W the following primers and auxiliary oligonucleotides are used:

For SNP C176F

| | |
|---|---|
| PCR primers | |
| Primer 27.6 | |
| 5′ GAT GGA TGA CGG AGG TTG TGA | (SEQ ID NO: 6) |
| GGC GCT 3′ | |
| Primer 26.5′p | |
| 5′ P-ATA GGA TGG CAG CGC TCA TGG | (SEQ ID NO: 7) |
| TGG GG 3′ | |
| Auxiliary oligonucleotides | |
| (24.5′) | |
| 5′ GCC TCA CAA CCT CCG TCA TCC | (SEQ ID NO: 8) |
| ATC 3′ | |
| (26.5′) | |
| 5′ ATA GGA TGG CAG CGC TCA TGG | (SEQ ID NO: 9) |
| TGG GG 3′ | |

For SNP S241F
Same primers as in Example 3, above: primer 26.1′P (SEQ ID NO: 1) and primer 27.2 (SEQ ID NO: 2).
For SNP R273H

PCR Primers

| | |
|---|---|
| Primer 27.4 | |
| 5′ ATA GGA TGA CGG AAC AGC TTT | (SEQ ID NO: 10) |
| GAG GTG 3′ | |
| Primer 26.3′p | |
| 5′ P-ATA GGA TGC CAG GAC AGG CAC | (SEQ ID NO: 11) |
| AAA CA 3′ | |
| Auxiliary oligonucleotides | |
| (24.3′) | |
| 5′ CTC AAA GCT GTT CCG TCA TCC | (SEQ ID NO: 12) |
| TAT 3′ | |
| (26.3′) | |
| 5′ ATA GGA TGC CAG GAC AGG CAC | (SEQ ID NO: 13) |
| AAA CA 3′ | |

The enzymatic reactions and assay are carried out as in Example 3, above. It is observed that SNPs that are so closely spaced that their primer sites overlap cannot be amplified in the same tube.

### Example 5

### Linear RCA Amplification of DNA to Produce Single Strand Fragments of Defined Size

The following example presents the use of a synthetic target which is phosphorylated at the 5′ end, if using lambda exonuclease, or unmodified if using T7 exonuclease.

Target DNA sequence 30. IP:

| | |
|---|---|
| 5′ P-CAG CTT TGA GGT GCG TGT TTG | (SEQ ID NO: 14) |
| TGC CTG TCC 3′ | |

is hybridized to padlock probe sequence 70. 1P:

| | |
|---|---|
| 5′ P-GCA CCT CAA AGC TGC GCA TCC | (SEQ ID NO: 15) |
| CAT CAG ATA GCG AGT CGA CGT GAG | |
| GAT GTA CGT GGA CAG GCA CA AAC | |
| AC 3′ | |

The padlock probe sequence 70.1P (SEQ ID NO: 15) has a region of complementarity to the target sequence (SEQ ID NO 14), in addition to Fok I restriction sites spanning the target sequence and a nonhomologous sequence that completes the padlock and contains primer recognition sites for a strand displacing polymerase such as phi29 DNA polymerase. The ligation and polymerization process are described, for example, by Zhong et al., (2001, Proc. Natl. Acad. Sci. 98:3940-3945). The target DNA sequence 30.1 P (SEQ ID NO; 14) and padlock probe 70.1P (SEQ ID NO: 15) are hybridized in 1×Taq DNA Ligase buffer (New England Biolabs, Beverly Mass.). Ligation proceeds at 45° C., 15 minutes with the addition of Taq DNA ligase. After heat inactivation at 70° C. for 10 minutes, the buffer is exchanged for exonuclease buffer by a size-exclusion column.

In the control reaction, target 30.2P

| | |
|---|---|
| 5′ P-CAG CTT TGA GGT GCC TGT TTG | (SEQ ID NO: 16) |
| TGC CTG TCC 3′ | |

is used, which contains a mismatch at the ligation site, such that the mismatch inhibits circularization of the padlock probe 70.1P (SEQ ID NO: 15). Alternatively, ligation can be inhibited by treating the padlock probe 70.1P with a phosphatase, or by using an unmodified version of the 70.1 sequence.

Addition of lambda exonuclease (New England Biolabs, Beverly Mass.) digests both the target sequence and the uncircularized probe. The circularized padlock probe remains intact (undigested by lambda exonuclease) and can be used as template for RCA. Primers that serve as template for the DNA polymerase are complementary to regions of the nonhomologous sequence of the circularized padlock. The product of the linear RCA is hybridized with auxiliary oligonucleotides

| | |
|---|---|
| 24.1 LOCK: | |
| 5′ ATG GGA TGC GCA GCT TTG AGG | (SEQ ID NO: 17) |
| TGC 3′ | |
| and | |
| 24.2 LOCK: | |
| 5′ TGT GCC TGT CCA CGT ACA TCC | (SEQ ID NO: 18) |
| TCA 3′ | |

which completes the double-stranded template for FokI digestion. The product that results from this reaction is a single-stranded 15-mer:

| | |
|---|---|
| 5′ GAG GTG CGT GTT TGT 3′. | (SEQ ID NO: 19) |

### Example 6

### Preparation of Single-Stranded DNA by a Nicking/Cleaving Strategy

A double-stranded PCR product is produced according to methods described herein. This method produces an oligomer having the desired nucleotide sequence, thereby generating a single stranded DNA molecule of defined sequence and length in accordance with the methods of the present invention. The double-stranded amplification product is incubated with a nicking enzyme and a cleavage enzyme, such that the double-stranded amplification product is nicked at one end of the defined sequence and cleaved at the other end of the defined sequence. In the present example, FokI binds to a recognition site on the exogeneous sequence introduced by one primer, and cuts at one end of the amplification product. The double-stranded amplification is nicked at the other end of the desired sequence.

The following primers can be used:

```
                N.BstNB I Nick Fok I cut
        (x) (16) ↓(5)(4) ↓ (10) ↓
5′-M(x)N(16)GAGTCNNNN*NNNNNNNNNN-----*---- --------- (SEQ ID NO: 20)
                                            ----------SWXYZ*
                                                                            NNNN*NNNNNNNNNGTAGGN(16)M(y)-5′
                                                                              (4)↑ (9)↑(5) (16) (y)
                                                                            Fok I cut Fok I site
                                                                                                                    (SEQ ID NO: 21)
```

In this example, N represents nucleotides in the primer that are the same as in the genomic DNA. S represents a single nucleotide polymorphism (SNP), and W, X, Y, and Z represent nucleotides in the genomic DNA that are not found in either primer. The nucleotides designated M, exogenous nucleotide sequence(s) not in the target genomic DNA, can be included in the primers to increase the length of the double helical products that remain after nicking/cleavage. The present example shows that x(M) nucleotides can be added to the top fragment, and y(M) nucleotides can be added to the lower fragment.

Following the nicking/cleaving reaction, the oligonucleotide having the defined sequence is 15 nucleotides long. The left hand fragment (top) primer strand is 25+x nucleotides long, and the left hand (lower) strand is 35+x long. The right hand fragment (lower) primer strand is 30+y long, and the upper strand is 34+y long. The melting temperature of these structures depends on the length of the shorter arm, 25 and 30 in this example.

In the present example, the primer strand is labeled with biotin at the 5′-end. For easy separation of the 15-mer having the defined sequence from the remainder of the amplification product, which includes its complement and the primer duplexes of the amplification product, the duplexes between the primer strand and the lower strand for the left primer (upper strand for the right primer) should remain intact when the 15-mer is melted from its complement. The nucleotides M on the left and right primers provide a mechanism for increasing the stability of these duplexes by increasing their lengths by amounts x and y respectively. In a multiplex mixture, all of the 15-mers having desired defined sequence should melt at lower temperature than any of the primer duplexes. The primer complexes are removed by attachment to magnetic beads carrying streptavidin that binds to biotin labels attached to the 5′ end of at least one primer. It may be desirable to add EDTA to the mixture to chelate Mg2+ in order to lower the stability of the 15-mer duplex to the stability range of the beads.

### Example 7

### Ligase Chain Reaction

Primer design for a typical LCR reaction:

Target Strand Sequence:

| | |
|---|---|
| 3′ ATTAGATGACCCTGCCTTGTCGAAACTCCACGCACAAACACGGACAGGACCCTCTCTGGCCGC 5′ | (SEQ ID NO: 22) |
| (P1) 5′ atgtaCAGCTTTGAGGTGCG | (SEQ ID NO: 23) |
| PTGTTTGTGCCTGTCagtga 3′ (P2) | (SEQ ID NO: 24) |

Bases complementary to the padlock probe are indicated in lower case. The P2 primer carries a 5′-phosphate residue. The SNP position is double-underlined.

Complementary-Target Strand Sequence:

| | |
|---|---|
| 5′ TAATCTACTGGGACGGAACAGCTTTGAGGTGCGTGTTTGTGCCTGTCCTGGGAGAGACCGGCG 3′ | (SEQ ID NO. 25) |
| (P4) 3′ GTCGAAACTCCACGP | (SEQ ID NO. 26) |
| CACAAACACGGACAG 5′ (P3) | (SEQ ID NO. 27) |

Padlock Probe Sequence:

| | |
|---|---|
| 5′-phos-GCACCTCAAAGCTGTACATCCTGCCA | (SEQ ID NO. 28) |
| GATTGCGAGTTGAATCACGGATGGTCACTGACAG | |
| GCACAAACAC-3′ | |

Regions of the padlock probe that are complementary to LCR probes P1 and P2 are underlined. The SNP base is at the 3′-end of the padlock probe sequence.

A typical LCR reaction contains 10-100 ng genomic DNA, 8-20 units Taq ligase, 50 ng salmon sperm DNA, and 150 nM LCR primers in a total volume of 20 μL Taq ligase buffer. Typical thermocycling conditions are 4 min at 94°, 4 min at 70°, and ˜30 cycles of denaturation (1 min at 90°) followed by ligation (1 min at 70°).

### Example 8

### Effect of High Concentrations of Genomic DNA on Ligase Chain Reaction

In this example, LCR was used to generate a product corresponding to a target nucleotide sequence using human genomic DNA (gDNA-Clontech) as a template. As positive controls, either a synthetic DNA sequence or a 540 base pair PCR product was used as the template nucleic acid.

The target sequence to be amplified by LCR was:

| | |
|---|---|
| 5′ GAC GGA ACA GCT TTG AGG TGC GTG | (SEQ ID NO: 29) |
| TTT GTG CCT GTC CTG GGA-3′. | |

A double stranded nucleic acid corresponding to this target sequence was generated synthetically for use as a positive control. This synthetic sequence was designated 42.1. As an additional positive control, a nucleic acid comprising the target nucleotide sequence of SEQ ID NO: 29 was generated by amplifying an approximately 540 base pair region of human genomic DNA using the following primer pair:

| | |
|---|---|
| 5′-TCT GAC TGT ACC ACC ATC C-3′; | (SEQ ID NO: 30) |
| and | |
| 5′-TTT CTT GCG GAG ATT CTC TTC | (SEQ ID NO: 31) |
| C-3′. | |

The primers used to amplify the target sequence by LCR were as follows:

| | |
|---|---|
| 23.4 | |
| 5′-CCG ACG GAA CAG CTT TGA GGT | (SEQ ID NO: 32) |
| GC-3′ | |
| 23.2 | |
| 5′-AGT CCC AGG ACA GGC ACA AAC | (SEQ ID NO: 33) |
| AC-3′ | |
| 21.6 | |
| 5′-GCA CCT CAA AGC TGT TCC GTC-3′; | (SEQ ID NO: 34) |
| and | |
| 21.7 | |
| 5′-GTG TTT GTG CCT GTC CTG GGA-3′. | (SEQ ID NO: 35) |

Primers 21.6 and 21.7 were both phosphorylated at the 5′ end. Additionally, primer 21.7 was end labeled with ³²P.

All amplification reactions were performed using 0.8 units of Pfu DNA ligase (Epicenter) in 20 μl of ligase buffer following the manufacturers instructions. Each reaction also 5 μM of each of the above primers. The template DNA provided in each reaction was as follows:
1) 100 μM of synthetic target 42.1 (SEQ ID NO: 29)
2) 100 ng of human genomic DNA
3) 800 ng of human genomic DNA
4) 800 ng of human genomic DNA supplemented with 0.2 mg/ml sonicated salmon sperm DNA
5) 540 bp PCR product
6) no target control

LCR was initiated by incubating each reaction at 94° C. for 4 minutes then at 67° C. for four minutes. Following the 67° C. incubation, the reactions were cycled between 91° C. for 1 minute and 67° C. for three minutes for 30 cycles.

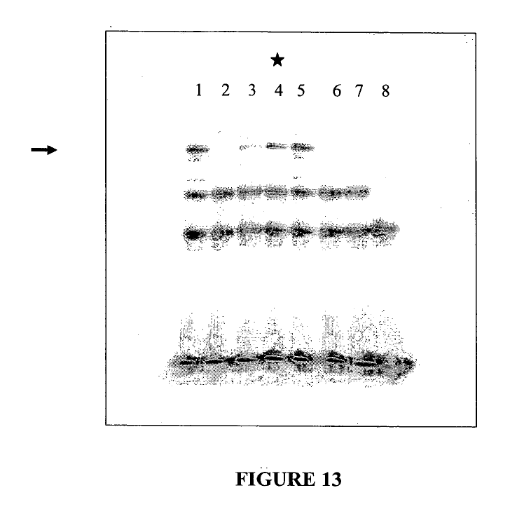

Following LCR samples of each reaction were loaded onto a native polyacrylamide gel and run in 1×TBE at 4° C. until the loading dye had run nearly the entire length of the gel. Bands corresponding to LCR product and labeled primers were visualized using autoradiography. FIG. 13 depicts an autoradiogram of a native polyacrylamide gel which shows that LCR product corresponding to the specifically targeted nucleotide sequence was produced from reactions containing a high concentration (800 ng) of genomic DNA (lanes 3 and 4, arrow). Furthermore, the addition of sonicated salmon sperm DNA at a concentration of 0.2 mg/ml increased production of the specific LCR product (lane 4).

### Example 9

### Hybridization of Padlock Probe to Target and Subsequent Ligation to Form a Catenated Duplex

This Example demonstrates that a padlock probe having a 5′ end and a 3′ end that is complementary to a single-stranded target nucleotide sequence can be ligated so as to form a catenated duplex with the single-stranded target nucleotide sequence.

The synthetic DNA target sequence 42.1 (SEQ ID NO: 29) was synthesized and used as the template nucleic acid containing the target nucleotide sequence. A single-stranded padlock probe was synthesized so as to comprise a backbone nucleotide sequence flanked at its 5′ end by a nucleotide sequence complementary to a portion of the target nucleotide sequence and flanked at its 3′ end by a nucleotide sequence complementary to the remainder of the target nucleotide sequence such that hybridization of the ends of the padlock probe with the target nucleotide sequence would place the 5′ end of the padlock probe immediately adjacent with the 3′ end of the padlock probe.

Padlock probe sequence:

| | |
|---|---|
| 5′-GCACCTCAAAGCTGTTCCGTC CCTGCCAGA | (SEQ ID NO: 36) |
| TTGCGAGTTGAATCACGGATGGTCCCAGGACAGG | |
| CACAAACAC-3. | |

Italics indicates backbone portion of the padlock probe.

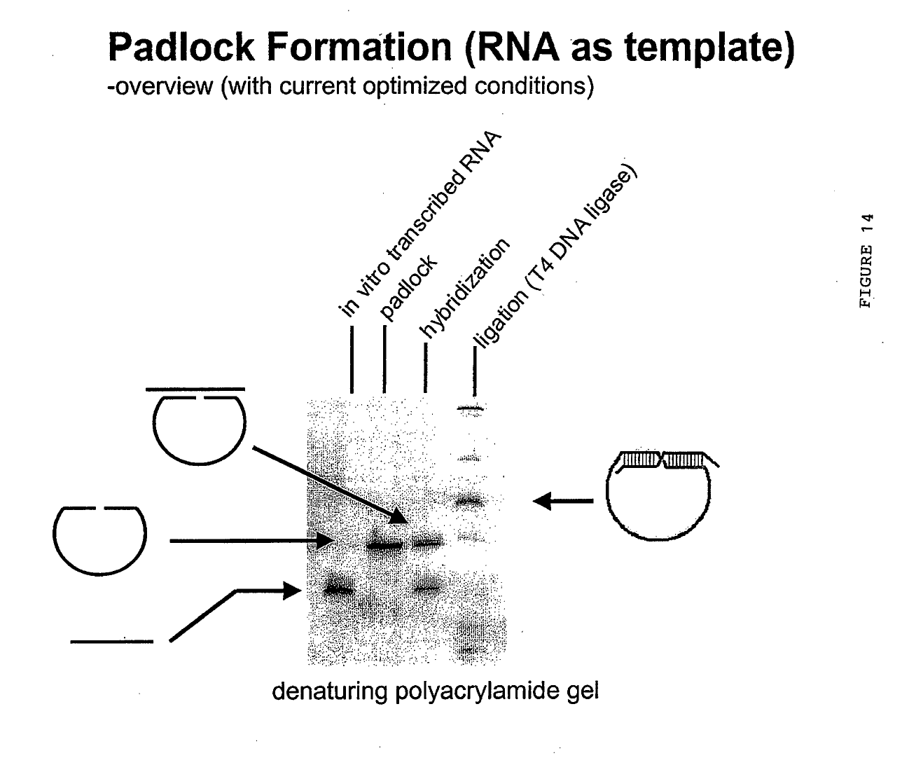

The synthetic DNA target nucleotide sequence was incubated with an approximately equal molar amount of padlock probe and allowed to hybridize at 67° C. in the presence of 8.0 units of Taq DNA ligase (New England Biolabs). Following the ligation, products of the reaction were analyzed on a denaturing polyacrylamide gel. FIG. 14 shows that ligation of the ends of the padlock probe causes the formation of a catenated duplex nucleic acid between the target RNA and the circularize padlock probe (lane 4).

### Example 10

### Amplification of a Target Nucleotide Sequence in a Template Nucleic Acid and Detection Using a POD Antibody Conjugate

This Example illustrates a method of detecting an LCR product that is first bound to a gene chip then amplified. This method can be applied to the detection of specific alleles in a genetic sample.

An allele-specific LCR product is selectively produced as described in Example 7 with the following modification. The LCR primer P1 contains an exogenous nucleotide sequence at its 5′ end that is complementary to the sequence of a capture probe. Thus, the allele-specific LCR product that is produced contains a specific sequence that permits hybridization of the LCR product to the capture probe.

Subsequent to LCR, the reaction mixture is incubated at 37° C. for 30 minutes with a capture probe that is attached to an electrochemical detection zone on a gene chip and which is complementary to the exogenous sequence in LCR primer P1. Following the incubation, the chip is washed with a buffer of 10 mM HEPS, 233 mM LiCl and 0.05% Tween 20 at pH 7.4 then rinsed with a buffer of 10 mM HEPS supplemented with 200 mM NaCl at pH 7.4. Both the wash and rinse steps are repeated. After washing and rinsing, only complete LCR products and primer P1 are bound to the capture probe.

Next, a padlock probe, which includes end sequences complementary to the LCR product (see Example 7) and an exogenous sequence that is identical to the sequence of T7-F2, a fluorescein-containing detection probe having the sequence 5′-CCTATAGTGAGTCGT-3′ (SEQ ID NO: 37), is incubated with the gene chip. The padlock probe is ligated and RCA is performed by adding to the chip dNTPs and φ29 polymerase (New England Biolabs) dissolved in φ29 polymerase buffer supplemented with 100 mM KCl. The RCA extension reaction is incubated for 1 hour at 37° C. then at room temperature for 30 minutes. The chip is washed twice with a buffer comprising 10 mM HEPS, 233 mM LiCl and 0.05% Tween 20 at pH 7.5 then rinsed with 10 mM Tris containing 200 mM NaCl. Since the padlock probe will only bind and form a circle with complete LCR products, bound PI primer sequence will not be amplified.

For detection, the chip is incubated with 0.25 μM of a fluorescein containing signal probe T7-F2, which has the sequence 5′-CCTATAGTGAGTCGT-3′ (SEQ ID NO: 37), in a hybridization buffer comprising 10 mM Tris, 1 M NaCl, 0.05% Tween 20 and 0.05% bovine serum albumin (BSA). Hybridization occurs at 37° C. for 10 minutes then at room temperature for an additional 30 minutes. Following the hybridization, the chip is washed with PBS comprising 0.05% Tween 20. Next, the chip is incubated for 20 minutes at room temperature with a 1:200 dilution of antifluorescein antibody conjugated to peroxidase (POD) in PBS buffer comprising 0.5% casein and 0.05% Tween 20. The chip is then washed with PBS containing 0.05% Tween 20 and signal is detected using K-blue TMB.

Only capture probes which have complete LCR products bound thereto produce substantial detectable signal.

### Example 11

### Amplification of a Target Sequence in a Template Nucleic Acid and Detection Using Ruthenium

This Example illustrates a method of detecting an LCR product that is first amplified then bound to a gene chip. As with the method described in Example 10, this method can be applied to the detection of specific alleles in a genetic sample.

An allele-specific LCR product is selectively produced as described in Example 7. In this case, the allele-specific LCR product that is produced does not contain an exogenous sequence or any other sequence that permits hybridization of the LCR product to the capture probe.

Subsequent to LCR, a padlock probe, which includes end sequences complementary to the LCR product (see Example 7) and an exogenous sequence that is identical to the sequence of a capture probe, is incubated with the LCR reaction mixture. The mixture is heated to an annealing temperature that permits binding of complete LCR products to the padlock probe but which excludes binding of single LCR primers. RCA is then performed by adding dNTPs and φ29 polymerase (New England Biolabs) dissolved in φ29 polymerase buffer supplemented with 100 mM KCl. The RCA extension reaction is incubated for 1 hour at the annealing temperature. Since the padlock probe will only bind and form a circle with complete LCR products, single LCR primers will not be amplified.

Subsequent to RCA, the reaction mixture is incubated at 37° C. for 30 minutes with a capture probe that is attached to an electrochemical detection zone on a gene chip and, which is complementary to the exogenous sequence in the single-stranded amplified RCA product. Following the incubation, the chip is washed with a buffer of 10 mM HEPS, 233 mM LiCl and 0.05% Tween 20 at pH 7.4 then rinsed with a buffer of 10 mM HEPS supplemented with 200 mM NaCl at pH 7.4. Both the wash and rinse steps are repeated. After washing and rinsing, only single-stranded RCA products are bound to the capture probe.

For detection, the chip is washed three times with a ruthenium detection solution containing 5 μM Ru(NH₃)₆³⁺, 10 mM Tris and 10 mM NaCl at pH 7.4. Binding of ruthenium to hybridized nucleic acids is measured using amperometry.

Only capture probes which have amplified RCA products bound produce substantial detectable signal.

It will be understood by those of skill in the art that numerous and various modifications can be made without departing from the spirit of the present invention. Therefore, it should be clearly understood that the forms of the present invention are illustrative only and are not intended to limit the scope of the present invention.

The disclosure of each reference cited above is incorporated herein by reference in its entirety.

1. A method for detecting a nucleic acid comprising:
hybridizing a first ligase chain reaction (LCR) primer and a second LCR primer to a target nucleotide sequence of a template nucleic acid such that the 3′ end of said first LCR primer is adjacent to the 5′ end of said second LCR primer;
ligating said first LCR primer to said second LCR primer to generate an LCR product;
hybridizing an RCA probe to at least a portion of said LCR product which includes the 3′ end of said LCR product;
initiating polymerization from said 3′ end of said LCR product, thereby generating an amplified single-stranded nucleic acid which comprises a plurality of LCR products; and
detecting said amplified single-stranded nucleic acid or fragments thereof.

2. The method of claim 1, wherein at least one LCR primer comprises an exogenous nucleotide sequence which is not complementary to said target nucleotide sequence.

3. The method of claim 2, wherein said exogenous nucleotide sequence is complementary to at least a portion of a nucleotide sequence present in a capture probe.

4. The method of claim 2, wherein said exogenous nucleotide sequence is complementary to at least a portion of a sequence present in a detection probe.

5. The method of claim 1, wherein said RCA probe comprises an exogenous sequence which is not present in said target sequence.

6. The method of claim 5, wherein said exogenous nucleotide sequence is identical to at least a portion of a nucleotide sequence present in a capture probe.

7. The method of claim 5, wherein said exogenous nucleotide sequence is identical to at least a portion of a sequence present in a detection probe.

8. The method of claim 1, wherein said RCA probe is circular.

9. The method of claim 1, wherein said RCA probe is a padlock probe.

10. The method of claim 9, further comprising ligating the ends of said padlock probe to one another, thereby generating a circularized padlock probe.

11. The method of claim 1, wherein detecting the amplified single-stranded nucleic acid comprises coupling one or more detector reagents to said amplified single-stranded nucleic acid and detecting said detector reagent.

12. The method of claim 1, wherein detection the amplified single-stranded nucleic acid comprises detecting an electrical signal indicative of the presence of said amplified single-stranded nucleic acid.

13. The method of claim 1, wherein said detector reagent is a detection probe.

14. The method of claim 13, wherein said detection probe is complementary to a nucleotide sequence that is present in the amplified single-stranded nucleic acid.

15. The method of claim 13, wherein a detector molecule is coupled to said detection probe.

16. The method of claim 15, wherein said detector molecule is fluorescein.

17. The method of claim 12, wherein said detector reagent is a redox enzyme.

18. The method of claim 17, wherein said redox enzyme is HRP.

19. The method of claim 12, wherein said detector reagent is a metal ion.

20. The method of claim 19, wherein said metal ion is ruthenium.

21. The method of claim 1, wherein said template nucleic acid comprises a single-stranded nucleic acid.

22. The method of claim 1, wherein said template nucleic acid comprises a double-stranded DNA.

23. The method of claim 22, wherein said double-stranded DNA is a genomic DNA.

24. The method of claim 22, wherein said first LCR primer comprises an exogenous nucleotide sequence located adjacent to the 5′ end of a nucleotide sequence which is complementary to a portion of a target nucleotide sequence and wherein the second LCR primer comprises an exogenous nucleotide sequence located adjacent to the 3′ end of a nucleotide sequence which is complementary to a portion of said target nucleotide sequence.

25. The method of claim 22, wherein said LCR primers comprise sequences complementary to variant sequences of said target nucleotide sequence, such that only LCR primers complementary to said variant sequence present in said target nucleotide sequence hybridize to said template nucleic acid and form at least one LCR product comprising sequence complementary to said variant sequence that is present in said target nucleotide sequence.

26. A kit comprising:
a chip having a capture probe attached thereto;
a detector reagent; and
instructions for using said detector reagent to detect an amplified target nucleotide sequence attached to said chip via said capture probe.

27. The kit of claim 26, wherein said capture probe is attached to a detection zone.

28. The kit of claim 26, wherein said detector reagent is a detection probe.

29. The kit of claim 28, wherein said detection probe is complementary to a nucleotide sequence that is present in the amplified single-stranded nucleic acid.

30. The kit of claim 28, wherein a detector molecule is coupled to said detection probe.

31. The kit of claim 30, wherein said detector molecule is fluorescein.

32. The kit of claim 26, wherein said detector reagent is a redox enzyme.

33. The kit of claim 32, wherein said redox enzyme is HRP.

34. The kit of claim 26, wherein said detector reagent is a metal ion.

35. The kit of claim 34, wherein said metal ion is ruthenium.

36. The kit of claim 26, further comprising an LCR primer pair.

37. The kit of claim 36, wherein at least one LCR primer of said primer pair comprises an exogenous nucleotide sequence that is complementary to at least a portion of a nucleotide sequence of said capture probe.

38. The kit of claim 36, wherein at least one LCR primer of said primer pair comprises an exogenous nucleotide sequence that is complementary to at least a portion of a nucleotide sequence of a detection probe.

39. The kit of claim 26, further comprising an RCA probe having an exogenous nucleotide sequence that is identical to at least a portion of a nucleotide sequence of said capture probe.

40. The kit of claim 39, wherein said RCA probe is a padlock probe.

41. The kit of claim 39, wherein said RCA probe is circular.

42. The kit of claim 26, further comprising an RCA probe having an exogenous nucleotide sequence that is identical to at least a portion of a nucleotide sequence of a detection probe.

43. The kit of claim 42, wherein said RCA probe is a padlock probe.

44. The kit of claim 42, wherein said RCA probe is circular.
